(19) United States
(12) Patent Application Publication
(10) Pub. No.: US 2023/0173113 A1
(43) Pub. Date: Jun. 8, 2023

(54) CYCLIC PEPTIDES AND THEIR CONJUGATES FOR ADDRESSING ALPHA-V-BETA-6 INTEGRIN IN VIVO

(71) Applicant: Technische Universität München, Munich (DE)

(72) Inventors: Johannes Notni, Sprockhövel (DE); Neil Quigley, München (DE); Katja Steiger, Hilgertshausen-Tandern (DE)

(73) Assignee: Technische Universität München, Munich (DE)

(21) Appl. No.: 17/910,728

(22) PCT Filed: Mar. 12, 2021
(86) PCT No.: PCT/EP2021/056424

§ 371 Date: Sep. 9, 2022

(30) Foreign Application Priority Data
Mar. 12, 2020 (EP) 20162699.1

(51) Int. Cl. A61K 51/08; A61P 35/00; C07K 7/64

(52) U.S. Cl. CPC A61K 51/082; A61P 35/00; C07K 7/64; A61K 51/088

(57) ABSTRACT

The invention provides conjugates of cyclic peptides as ligands for cellular surface receptors, in particular, as ligands for αvβ6-integrin. The conjugates further contain effector moieties and are suitable for use as therapeutic agent, diagnostic agent, agent for imaging, targeting moiety and as biomolecular research tool. The invention specifically relates to the use of conjugates with signalling moieties or radionuclides for in-vivo addressing of αvβ6-integrin.

### TECHNICAL FIELD

The invention relates to the field of cyclic peptides as ligands for cellular surface receptors, in particular, as ligands for αvβ6-integrin. It furthermore relates to conjugates of such peptides with effector moieties that are suitable for use as therapeutic agent, diagnostic agent, targeting moiety and biomolecular research tool. The invention specifically relates to the use of derivatives of such peptides with signalling moieties or radionuclides for in-vivo addressing of αvβ6-integrin.

### BACKGROUND

Integrins are a class of 24 heterodimeric cellular transmembrane receptors, all comprising one out of 18 α- and 8 β-subunits. They mediate the selective binding of cells to various extracellular matrix proteins, such as vitronectin, fibronectin, collagen, or laminin, and furthermore are involved in signalling pathways.¹ αvβ6 is one of 8 integrin subtypes that recognize the arginine-glycine-aspartate (RGD) peptide sequence. In contrast to other popular RGD-binding integrins, such as αvβ3 and α5β1, which are expressed by different cell types and have gained considerable attention due to their involvement in formation and sprouting of blood- and lymphatic vessels (vascularisation, angiogenesis and lymphangiogenesis),² αvβ6 integrin levels in adult tissues are generally low.³ Expression of αvβ6 integrin is restricted to epithelial cells.⁴ Accordingly, many tumors of epithelial origin (carcinomas) show an enhanced αvβ6 integrin expression,⁵ above all, pancreatic,⁶ but also cholangiocellular,⁷ gastric,^{8,9} breast,¹⁰ ovarian,^{11,12} colon,¹³ and those of the upper aerodigestive tract.¹⁴ αvβ6-integrin has furthermore been described as a marker for increased invasiveness and malignancy of several carcinomas and thus, poor prognosis.^{5,8,11,13} Hence, αvβ6-integrin has been proposed as a target for in-vivo addressing of carcinoma tissue for the purpose of molecular imaging and targeted therapy.¹⁵ Moreover, αvβ6-integrin is involved in the epithelial-mesenchymal transition (EMT), e.g., during development of biliary,¹⁶ renal,¹⁷ as well as pulmonary¹⁸ fibrosis, and thus might serve as a fibrosis marker.

### STATE OF THE ART

Several αvβ6-specific, non-peptidic¹⁹ as well as peptidic inhibitors^{20,21,22,23} have been reported. The linear peptides A20FMDV2,²¹ H2009.1,²² and the cyclic peptide S₀2²³ have been equipped with radiolabels and applied for in-vivo imaging of αvβ6-integrin expression²⁴ by single-photon emission computed tomography (SPECT)^{25,26,27} and positron emission tomography (PET).^{21,28,29,30,31,32} Recently, radiolabeled compounds targeting αvβ6-integrin were tested for imaging of carcinomas in humans.^{33,34,35}

The cyclic nonapeptide cyclo(FRGDLAFp(NMe)K)^{36,37} (herein abbreviated Phe₂) was reported to show a high affinity to αvβ6-integrin (0.26 nM), a remarkable selectivity against other integrins (αvβ3: 632 nM; α5β1: 73 nM; αvβ5 and αIIbβ3: >1 μM), and full stability in human plasma up to 3 h. Derivatives of Phe₂ were equipped with various chelators for radiometal binding^{38,39} and their in-vivo properties evaluated in tumor-bearing mice. These investigations have shown that radiolabelled chelator conjugates comprising only one Phe₂ moiety (monomers) show relatively low uptake in the αvβ6-expressing tumor tissue.³⁹ Conjugates comprising two and particularly three Phe₂ moieties (dimers and trimers, respectively) showed higher affinities to αvβ6-integrin, but were also characterized by relatively high levels of unspecific uptake in non-target organs because of their lipophilicity. This behaviour of the trimers could not be mitigated by introduction of pharmacokinetic modifiers, namely hydrophilic PEG linkers.³⁸

### SUMMARY OF THE INVENTION

With regard to the above described situation, there is a need for providing αvβ6-integrin active functionalized compounds with improved pharmacokinetics and especially an increased target-specific tissue uptake and retention accompanied by low unspecific uptake in αvβ6-integrin negative tissues. In particular, a low unspecific uptake in liver tissue and pancreatic tissue is desirable. Further objectives are rapid clearance from the blood pool and a low unspecific binding to blood components, as well as suitability for high-contrast in-vivo imaging of such tissues, expressed as high ratios of uptakes in tumor lesions over other tissues.

The present invention solves this problem by providing conjugates comprising specific cyclic nonapeptides targeting αvβ6-integrin. These cyclic nonapeptides are characterized by the following amino acid sequences: cyclo(YRGDLAYp(NMe)K), hereinafter termed Tyr₂, cyclo(FRGDLAYp(NMe)K), hereinafter termed FRGD, and cyclo(YRGDLAFp(NMe)K), hereinafter termed YRGD. These abbreviations are also used to characterize the respective cyclopeptides being covalently bonded to an effector moiety via the terminal amino group of the (NMe)K sidechain. This means, in other words, that the abbreviations Tyr₂, FRGD and YRGD characterize not only the cyclopeptides cyclo(YRGDLAYp(NMe)K), cyclo(FRGDLAYp(NMe)K) and cyclo(YRGDLAFp(NMe)K), respectively, but also the same cyclopeptides being in a form wherein one of the two hydrogens at the terminal amino group of the (NMe)K sidechain is absent/replaced by a covalent bond to another moiety.

Tyr₂, FRGD and YRGD are structurally related to Phe₂ and they are all encompassed by the general teaching of patent application WO 2017/046416 A1. However, this patent application does not specifically disclose Tyr₂ and it also does not disclose any specific conjugates with Tyr₂, FRGD and/or YRGD and/or any tissue specific binding characteristics of conjugates comprising Tyr₂, FRGD and/or YRGD.

Surprisingly, it was found that conjugates of Tyr₂, FRGD and/or YRGD, in particular those containing more than one Tyr₂, FRGD and/or YRGD moiety, show a high target-specific tissue uptake and retention, accompanied by low unspecific uptake in αvβ6-integrin negative tissues (particularly, the liver) and a rapid clearance from the blood pool, as compared to, e.g., structurally equivalent derivatives of Phe₂. Hence, such conjugates allow for selective and specific addressing of αvβ6-integrin positive tissues in vivo, in particular, for high-contrast in-vivo imaging of such tissues.

The invention thus relates to conjugates of Tyr₂, FRGD and/or YRGD wherein an effector moiety is covalently attached to the terminal amino group of the NMe-lysine residue or at least one cyclic nonapeptide selected from Tyr₂, FRGD and YRGD. The invention relates in particular to conjugates comprising more than one Tyr₂, FRGD and/or YRGD moiety, which exhibit higher affinities and integrin subtype selectivities than comparable compounds comprising only one such moiety. These conjugates can be characterized by the following general formula (I):

E(Cp)ₙ (I)

wherein Cp represents a cyclopeptide selected from Tyr₂, FRGD and/or YRGD, n is an integer selected from 1 to 4 and E represents the effector moiety.

According to the invention, various types of effector moieties can be used, including moieties suitable for diagnostic uses, as well as pharmacologically active moieties for therapeutic uses. Of particular interest are conjugates with moieties for diagnostic uses. These include moieties comprising radionuclides (for nuclear imaging or radioguided surgery), fluorophores (for fluorescence imaging or fluorescence-guided surgery), or signalling units for magnetic resonance imaging (MRI). For therapeutic purposes, the effector moiety may for instance contain a radionuclide (endoradiotherapy) or chemotherapeutic agent (targeted drug delivery).

Yet another aspect of the present invention pertains to uses of the above-mentioned conjugates in diagnostic methods or therapeutic methods.

The cyclopeptide Tyr₂ is novel. Building blocks containing one of Tyr₂, FRGD and YRGD combined with a spacer element adapted for Click chemistry couplings are also novel. Another aspect of the present invention therefore relates to the provision of these compounds.

The various aspects of the present application are described in more detail in the detailed description below and in the appended claims.

### DESCRIPTION OF FIGURES

FIG. 1: Exemplary positron emission tomography (PET) scans (maximum intensity projections) of the same H2009 tumor-bearing SCID mouse, 75 min after injection of Ga-68-TRAP(Phe₂)₃ (left) and Ga-68-C-7 (right). The time between both scans was 24 h.

FIG. 2: Ex-vivo biodistribution of Ga-68-TRAP(Phe₂)₃ (structured bars) and Ga-68-C-7 (plain bars) in H2009 tumor-bearing SCID mice, 90 min p.i., without blockade (approx. 0.1 nmol, n=5) and with blockade (50 nmol, n=3) (data expressed as mean±standard deviation).

FIG. 3: Biokinetics of Ga-68-TRAP(Phe₂)₃ (left) and Ga-68-C-7 (right), obtained from a region-of-interest based evaluation of 90-min dynamic PET scans.

FIG. 4: Top: ex-vivo biodistribution in selected tissues of H2009 tumor-bearing SCID mice, 90 min p.i., without blockade (approx. 0.1 nmol, n=5) and with blockade (50 nmol, n=3) Bottom: Tumor-to-tissue ratios derived from biodistribution data. All data expressed as mean±standard deviation. Key to column labels: a) Ga-68-TRAP(Phe₂)₃; b) Ga-68-TRAP(Phe₂)₃, blockade; c) Ga-68-C-11; d) Ga-68-C-11, blockade; e) Ga-68-C-9; f) Ga-68-C-9, blockade; g) Ga-68-C-8; h) Ga-68-C-8, blockade; i) Ga-68-C-10; k) Ga-68-C-10, blockade; 1) Ga-68-C-7; m) Ga-68-C-7, blockade.

FIG. 5: Exemplary positron emission tomography (PET) scans (maximum intensity projections) of the same H2009 tumor-bearing SCID mouse, 75 min after injection of Ga-68-TRAP(Phe₂)₃, Ga-68-C-9, Ga-68-C-8, and Ga-68-C-7 (from left to right). The time between the scans was 24 h. % IA/mL means percent of injected activity per mL tissue.

FIG. 6: Biokinetics of Ga-68-TRAP(Phe₂)₃, Ga-68-C-9, Ga-68-C-8, and Ga-68-C-7 (from left to right), obtained from a region-of-interest based evaluation of 90-min dynamic PET scans. % IA/mL means percent of injected activity per mL tissue.

### DETAILED DESCRIPTION

### Definitions

The term “derived from” indicates that an atomic group contained in the conjugate has the same structure as the compound from which it is derived, the only difference being the replacement of a hydrogen atom by a covalent bond for binding the atomic group to the remainder of the conjugate.

The term “heavy atom” is used herein to characterize any atom other than hydrogen, deuterium or any other isotope thereof. In case of a bivalent atomic group, there must be at least one heavy atom with at least two free valences. If a heavy atom with more than two free valences is present, the remaining valences may be saturated by hydrogen or other heavy atoms.

Unless specified otherwise, standard amino acid nomenclature is used. Unless specified otherwise, amino acids are L-stereoisomers. Unless specified otherwise, amino acid moieties are linked to each other via peptide bonds. Unless specified otherwise, standard one-letter or three-letter code for amino acids applies. Unless specified otherwise, lower case letters indicate that the amino is in the D-configuration while upper case letters indicate that the amino acid is in the L-configuration.

Me refers to a methyl group. N-Me-amino acid refers to a group, wherein the α-amino group carries a methyl group.

Unless specified otherwise or the context dictates otherwise, references to the “compound of the invention”, “conjugate of the invention” or the like are to be understood as references not only to the compound, conjugate, etc. of the present invention as described hereinbelow and/or as specified in the appended claims, but also as references to the pharmaceutically acceptable salts, esters, solvates, and polymorphs thereof.

Reference to “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more. Substituents may be selected from alkyl, preferably C₁₋₆-alkyl, alkenyl, preferably C₂₋₆-alkenyl, alkynyl, preferably C₂₋₆-alkynyl, alkoxy, preferably C₁₋₆-alkoxy, acyl, preferably C₂₋₆-acyl, amino (including simple amino, mono and di-C₁₋₆-alkylamino, mono and di-C₆₋₁₄-arylamino, and C₁₋₆-alkyl-C₆₋₁₄-arylamino), C₂₋₆-acylamino (including carbamoyl, and ureido), C₁₋₆-alkylcarbonyloxy, C₆₋₁₄-arylcarbonyloxy, C₁₋₆-alkoxycarbonyloxy, C₁₋₆-alkoxycarbonyl, carboxy, carboxylate, aminocarbonyl, mono and di-C₁₋₆-alkylaminocarbonyl, cyano, azido, halogen, hydroxyl, nitro, trifluoromethyl, thio, C₁₋₆-alkylthio, arylthio, C₁₋₆-alkylthiocarbonyl, thiocarboxylate, C₄₋₈-cycloalkyl, heterocycloalkyl with 4 to 8 ring members, C₆₋₁₄-aryl, heteroaryl with 5 to 6 ring members optionally condensed with 1 or 2 saturated, unsaturated or aromatic carbocycle or heterocycle each having 5 or 6 ring members, C₆₋₁₄-aryloxy, C₆₋₁₄-aryloxycarbonyloxy, benzyloxy, benzyl, sulfinyl, C₁₋₆-alkylsulfinyl, sulfonyl, sulfate, sulfonate, sulfonamide, phosphate, phosphonato, phosphinato, oxo, guanidine, imino, formyl and the like. Any of the above substituents can be further substituted if permissible, e.g. by one or more of the listed substituent groups.

The terms “alkyl”, “alkenyl”, “alkynyl”, “cycloalkyl”, “carbocyclic”, “heterocycloalkyl”, “aryl”, “heteroaryl”, “heterocycle”, “amine”, “amide”, “nitro”, “halogen”, “thiol”, “hydroxyl” or “hydroxy”, “alkylthio”, “alkylcarboxyl”, “carbonyl”, “carboxy”, “acyl”, “solvate”, “pharmaceutically acceptable salt”, “pharmaceutically acceptable vehicle”, “pharmaceutically acceptable carrier” and “pharmaceutical composition” may have the meanings defined in WO 2017/046416 A.

Unless specified otherwise, all abbreviations are intended to have their commonly used meaning as represented, for instance, by the IUPAC-IUP Commission on Biochemical Nomenclature in Biochemistry 11, 1972, 942-944. For the atoms contained in the conjugate standard rules for the valences of apply, for instance as described in the Wikipedia entry “Valence (chemistry)” in the version of Jan. 24, 2020. Unless specified otherwise or the context dictates otherwise, if an atom has more valences as the shown number of bonding partners, the remaining valences are saturated by hydrogen atoms.

Unless specified otherwise, conjugates and other compounds of the present invention are “pharmaceutically acceptable” which means that the respective compounds are suitable for use with humans and/or animals without causing adverse effects (such as irritation or toxicity), commensurate with a reasonable benefit/risk ratio.

The term “or” is generally employed in its sense including “and/or” unless the content dictates otherwise.

“Room temperature” can be any temperature from 20° C. to 25° C. and preferably it is 22° C.

“Ga-68-TRAP(Phe₂)₃” refers to a compound previously described as “Ga-68-TRAP(AvB6)₃” by Maltsev et al.³⁸

Unless specified otherwise, the term “chelating group”, “chelator” or the like refers to a group that is capable to forming two or more, preferably three, four, five, six, seven or eight, coordinative bonds to a metal ion.

### Cyclopeptide

The Cyclopeptides used in the present invention are shown below:

Tyr₂: cyclo(YRGDLAYp(NMe)K),

FRGD: cyclo(FRGDLAYp(NMe)K),

YRGD: cyclo(YRGDLAFp(NMe)K)

### Conjugate

### General Structure

The general structure of the conjugates of the present invention may be characterized by the following formula (I)

E(Cp)ₙ (I)

wherein each Cp represents a cyclopeptide independently selected from Tyr₂, FRGD and/or YRGD, n is an integer selected from 1 to 4, preferably from 2 to 4 and more preferably 3 or 4, and E represents an effector moiety. According to a further embodiment, it is possible to use a polymeric or dendritic effector moiety. In this case, n may be an integer selected from 2 to 100, preferably 10 to 30. Suitable polymeric scaffolds include polyethyleminines, polysaccarides, polyamides, polypeptides, poly(amidoamine) (PAMAM) dendrimers, polypropylene imine) (PPI) dendrimers, polyether-copolyester (PEPE) dendrimers, polyether dendrimers, polyester dendrimers, and polyaryl ether dendrimers.

The one or more cyclopeptides are each covalently bonded to the effector moiety via the terminal amino group in the sidechain of the NMe-Lys residue.

In preferred embodiments, the conjugate of formula (I) contains 2, 3 or 4 cyclopeptide moieties. Most preferably, the conjugate of formula (I) contains 3 or 4 cyclopeptide moieties.

In the conjugates of the present invention containing two or more cyclopeptide moieties, these multiple cyclopeptide moieties may be the same or different from each other. All of the following specific conjugates are encompassed by the scope of the present invention:

E(Tyr₂)₁,E(Tyr₂)₂,E(Tyr₂)₃,E(Tyr₂)₄,

E(FRGD)₁,E(FRGD)₂,E(FRGD)₃,E(FRGD)₄,

E(YRGD)₁,E(YRGD)₂,E(YRGD)₃,E(YRGD)₄,

E(Tyr₂)₁(FRGD)₁,E(Tyr₂)₂(FRGD)₁,E(Tyr₂)₁(FRGD)₂,E(Tyr₂)₂(FRGD)₂,E(Tyr₂)₁(FRGD)₃,E(Tyr₂)₃(FRGD)₁,

E(Tyr₂)₁(YRGD)₁,E(Tyr₂)₂(YRGD)₁,E(Tyr₂)₁(YRGD)₂,E(Tyr₂)₂(YRGD)₂,E(Tyr₂)₁(YRGD)₃,E(Tyr₂)₃(YRGD)₁,

E(FRGD)₁(YRGD)₁,E(FRGD)₂(YRGD)₁,E(FRGD)₁(YRGD)₂,E(FRGD)₂(YRGD)₂,E(FRGD)₁(YRGD)₃,E(FRGD)₃(YRGD)₁,

E(Tyr₂)₁(FRGD)₁,(YRGD)₁,E(Tyr₂)₂(FRGD)₁,(YRGD)₁,E(Tyr₂)₁(FRGD)₂(YRGD)₁,E(Tyr₂)₁,(FRGD)₁(YRGD)₂

In case of polymeric or dendritic effector moieties, it is also possible to attach multiple copies of the same cyclopeptide selected from Tyr₂, YRGD and FRGD. Alternatively, the polymeric or dendritic effector may bind to two or three of these different cyclopeptides such that each of these two or three cyclopeptides is present one or more times with the proviso that the total number of bonded cyclopeptides is within the ranges specified above for n, i.e. the polymeric or dendritic conjugate is characterized by a general formula E((Tyr₂)ₙ₁(FRGD)ₙ₂(YRGD)ₙ₃) wherein each of n1, n2 and n3 may be in the range of from 0 to n with the proviso that n1+n2+n3=n.

It is, in principle, possible to obtain further compounds of the present invention by modifying a compound of the invention as specified above by covalently attaching cyclopeptides different from to Tyr₂, FRGD and YRGD to the effector moiety. For instance, one embodiment relates to compounds as described above, but wherein one, two or three of the cyclopeptide moieties Tyr₂, FRGD and/or YRGD is/are replaced by the cyclopeptide moiety Phe₂ mentioned in the introduction, wherein Phe₂ is linked to the remainder of the conjugate in the same way as the other cyclopeptide moieties, i.e. via the terminal amino group of the (NMe)K residue, and wherein the number of replacements by Phe₂ is such that at least one of the cyclopeptide moieties Tyr₂, FRGD and YRGD remains in the conjugate (i.e. if n is the number of cyclopeptide moieties, the number of Phe₂ moieties is no more than n−1 while at least one cyclopeptide moiety is selected from Tyr₂, FRGD and YRGD). In another embodiment, no further cyclopeptides are present.

### Effector Moiety

The effector moiety is an atomic group having from 10 to 1000 heavy atoms, preferably from 20 to 200 heavy atoms and more preferably from 30 to 150 heavy atoms. It is further characterized by the following characteristics:
(a) it has a number of free valences that corresponds to the number of bonded cyclopeptides, i.e. the number n in formula (I);
(b) it contains an active atom or group of atoms that is capable of exercising the desired effect, e.g., a radioisotope or chromophore for diagnostic purposes or a therapeutically active moiety for therapeutic purposes;
(c) it contains one or more groups of atoms acting as spacer to spatially separate the one or more cyclopeptides from the from the active atom or active group of atoms to thereby reduce mutual interference.

The effector may in some embodiments be characterized by the following general formulae (II) and (II′).

Aa(Cg)(S)ₙ (II)

Aa′(Cg)ₖ(S)ₙ (II′)

wherein Aa stands for an active atom or active atomic group that is capable of being bonded via chelation, Aa′ stands for an active atom or active atomic group that is capable of being bonded via covalent bonding, Cg stands for a chelating group, k is 0 or 1, S stands for an atomic group acting as a spacer and n is as defined above with respect to formula (I) with the proviso that n does not exceed the number of free valences of the chelating group and with the proviso that n is 1 if k is 0, i.e. that a single spacer is directly bonded to the active atom or active atom group if no chelating group is present. Combining formula (II) with formula (I) yields the following formula (Ia):

Aa(Cg)(SCp)ₙ (Ia)

wherein Aa, Cg, S, Cp and n have the same meanings as defined above with respect to formulae (I) and (II).

In a related embodiment, an active atom or active atomic group Aa′ is covalently bonded to the chelating group or to the spacer. The conjugate of this embodiment is characterized by the following formula (Ia′):

Aa′(Cg)ₖ(SCp)ₙ (Ia′)

wherein Aa′ is an active atom or active atomic group capable of forming covalent bonds, Cg, S, Cp and n have the same meanings as defined above with respect to formulae (I) and (II); k is 0 or 1; if k is 1, Aa′ is covalently bonded to Cg; if k is 0, Aa′ is covalently bonded to S. In this case, n is 1, i.e. there is only a single spacer which forms covalent bonds to Aa′ and Cp.

In another embodiment, it is possible to attach a second active atomic group to one of the spacers (instead of one of the cyclopeptides), such that the conjugate is represented by the following formula (Ib):

Aa(Cg)(SCp)_{n′}(SAa′) (Ib)

wherein Aa, Cg, S and Cp have the same meanings as in formula (Ia) above, and wherein Aa′ is an active atom or active atomic group different from Aa insofar as it is covalently bonded to the spacer and not via a chelating group, n′ is 1, 2 or 3 with the proviso that n′+1 is the number of free valences of the chelating group or less.

In yet another embodiment, different linkers may be connected via a non-chelating central moiety. In these cases, the active atom or active atomic group is covalently bonded to another part of the molecule, which can be either the central moiety, a spacer or a cyclopeptide. The conjugate of this embodiment is characterized by the following formulae (Ic), (Id), (Ie) and (1f):

Aa′(Cm)ₖ(SCp)ₙ (Ic)

(Cm)(SCp)ₙ₋ₒ(S(Aa′)ₚ(Cp)ₘ)ₒ (Id)

(Cm)(SCp)ₙ₋ₒ(SCp(Aa′)ₚ)ₒ (Ie)

Cp(Aa′)ₚ (If)

The formula (Ic) corresponds to the above formula (Ia′), but wherein the chelating group is replaced by a central moiety Cm. S, Cp and n have the same meanings as defined above with respect to formulae (I), (Ia), and (II); k is 0 or 1. Aa′ is an active atom or active atomic group that is capable of forming covalent bonds. In formula (Ic), it is bonded via a covalent bond to Cm if k is 1 and it is bonded to S if k is 0. In the latter case, n must be 1, i.e. there is only a single spacer binding both Aa′ and Cp.

The central moiety Cm can be any atom or atomic group having at least n+1 valences to accommodate n spacer-cyclopeptide moieties and 1 active atom or active atomic group. Cm preferably has 1 to 30 atoms selected from C, N, O, S and P. The remaining valences are saturated by hydrogen. Preferred Cm groups are aromatic groups such as phenyl, naphthyl, or derived from larger condensed aromatic groups containing 3 or 4 6-membered rings such as anthracen, phenantren, benzpyrene, etc.; nonaromatic cyclic groups including C5-7 carbocycles such as cyclopentane, cyclohexane, cycloheptane, condensed groups containing 2, 3 or 4 rings, each consisting of 5 to 7 ring members such as fully or partially hydrogenated forms of naphthalene, anthracen, phenantren, benzpyrene, etc., bi- or tricyclic groups having 7 to 10 carbon atoms such as norbornene or adamantane. Further preferred central moieties may be heterocyclic groups containing 1, 2, 3 or 4 condensed rings each having a ring size independently selected from 5, 6 or 7 ring members. These groups may be aromatic, partially or fully saturated. Alternatively, the central moiety may be a single atom selected from C, N and P.

Formula (Id) characterizes conjugates, wherein the cyclopeptide moieties and the active atom or active atomic group Aa′ are all linked to the central moiety via spacers. That is, the active atom or active atomic group Aa′ is covalently bonded to one of the spacers. The meanings of Cm, Aa′, S, Cp and n are the same as explained above with respect to formula (I), (II), (Ia) and (Ic). Optionally, the spacer carrying the active atom or active atomic group Aa′ may additionally carry a cyclopeptide Cp; hence, m may be 0 or 1. If an additional Cp is present, the active atom or active atomic group Aa′ and its point of attachment must be selected such that detrimental interactions with the cyclopeptide are avoided or at least minimized, e.g. by attaching the two moieties to different atoms of the spacer, which are at least 5 covalent bonds apart from each other. The number of spacers carrying the active atom or group Aa′ is characterized by o and it can be any integer from 1 to n. The number of active atoms Aa′ bonded to an individual spacer is characterized by p and it can be 1 or 2.

Formula (Ie) is characterized by the active atom or active atomic group Aa′ being bonded to the cyclopeptide Cp. The meanings of Cm, Aa′, S, Cp and n are the same as explained above with respect to formula (I), (II), (Ia) and (Ic). The variable o indicates the number of cyclopeptides Cp carrying an active atom or group Aa′. This can be any integer from 1 to n. The variable p characterizes the number of active atoms Aa′ bonded to an individual cyclopeptide and it can be 1 or 2.

Formula (If) characterizes conjugates of the present invention, which do not contain any central moiety and/or spacer. Instead, the active atom or active group Aa′ is directly bonded to the cyclopeptide. According to a preferred embodiment of formula (If), an iodine atom or radioisotope is attached to a 3-position of one or both of the tyrosine residues present in Tyr₂, FRGD or YRGD, so that the resulting cyclopeptides are cyclo(3-I-YRGDLAYp(NMe)K); cyclo(3-I-YRGDLA3-I-Yp(NMe)K); cyclo(YRGDLA3-I-Yp(NMe)K); cyclo(3-I-YRGDLAFp(NMe)K); cyclo(FRGDLA3-I-Yp(NMe)K);

wherein 3-I-Y represents a Tyr residue that carries an iodine atom in the 3-position of the phenyl ring, wherein said iodine atom can be any non-radioactive isotope or radioisotope of iodine.

The compounds of formula (1f) may have a dual character: as long as the binding of Aa′ does not lead to significant deterioration of the affinity to αvβ6-integrin, i.e. binding affinity of the Aa′-carrying cyclopeptide being 5 nM or lower when determined in accordance with the methods described in references 36 and 37, they may serve as conjugates of the present invention. In addition, they may also be incorporated into larger conjugates, e.g. of formula (1e), and thus serve as a building block of the invention.

The modes of binding active atoms and active groups Aa and Aa′ described above by means of formulae (1a) to (1f) may be freely combined. For instance, a compound of formula (1a) or (1a′) may carry one or more cyclopeptides which themselves carry one or more active atoms or active groups Aa′. In particular, the present invention also relates to conjugates of formula (1a) or (1a′), wherein one or more of the cyclopeptides carries one or two iodine atoms or radioisotopes bonded to the 3-position of tyrosine residues.

The active atom or active atomic group Aa, Aa′ may include the following:
(b-1) a non-radioactive isotope or a radioisotope of a metal ion selected from La³⁺, Ce³⁺, Pr³⁺, Nd³⁺, Sm³⁺, Eu²⁺, Gd³⁺, Tb³⁺, Dy³⁺, Ho³⁺, Er³⁺, Tm³⁺, Yb³⁺, Lu³⁺, Sc³⁺, Y³⁺, Ga³⁺, Fe³⁺, Co²⁺, Co³⁺, Ge⁴⁺, In³⁺, Sn⁴⁺, Bi³⁺, Rh³⁺, Ru³⁺, Ru⁴⁺, Ag⁺, Au³⁺, Pb²⁺, Pd³⁺, Pd⁴⁺, Pm³⁺, Ac³⁺, Ti⁴⁺, Zr⁴⁺, Al³⁺, Cr³⁺, Cu²⁺, Zn²⁺ and mixtures thereof. Particularly preferred is a metal ion selected from the group consisting of Ga³⁺, Gd³⁺, Cu²⁺, Sc³⁺, Y³⁺, and Lu³⁺ and mixtures thereof. The radioisotope may specifically be selected from ⁴³Sc, ⁴⁴Sc, ⁴⁶Sc, ⁴⁷Sc, ⁵⁵Co, ^{99m}Tc, ²⁰³Pb, ²¹²Pb, ⁶⁶Ga, ⁶⁷Ga, ⁶⁸Ga, ⁷²As, ¹¹¹In, ^{113m}In, ^{114m}In, ⁹⁷Ru, ⁶²Zn, ⁶¹Cu, ⁶²Cu, ⁶⁴Cu, ⁵²Fe, ^{52m}Mn, ⁵¹Cr, ¹⁸⁶Re, ¹⁸⁸Re, ⁷⁷As, ⁸⁶Y, ⁹⁰Y, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁵²Tb, ¹⁵⁵Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ⁸⁸Zr, ⁸⁹Zr, ²¹²Bi, ²¹³Bi, ²²⁵Ac and mixtures thereof, wherein these radioisotopes are preferably used in the form of metal ions in the respective oxidation states listed above. Particularly preferably the radioisotope is selected from the group consisting of ⁶⁸Ga, ⁴⁴Sc, ^{99m}Tc, ¹¹¹In, ⁶⁴Cu, ⁸⁹Zr, ⁹⁰Y, ¹⁷⁷Lu, ²¹³Bi, ²²⁵Ac and mixtures thereof.
(b-2) a non-metal radioisotope which is selected from ¹¹C, ¹³N, ¹⁵O, ¹⁸F, ¹²³I, ¹²⁴I, ¹²⁵I, or ¹³¹I, preferably ¹⁸F or ¹²³I. In addition to its presence as Aa, Aa′ or part thereof in the above formulae, said non-metal radioisotope may also be an active atom Aa′ that is present anywhere else within the molecule, where it may replace any other covalently bonded atom that already exists as part of the remaining molecule and which has an appropriate number of bonding partners.
(b-3) a chromophore of a fluorescence or non-fluorescent dye and preferably moieties derived from ThermoFisher's commercially available Cy® series such as CY® 3, 5, 5.5, 7, 7.5 and the AlexaFluor® series such as AlexaFluor® 350, 405, 488, 532, 546, 555, 568, 594, 647, 680, and 750 as well as Fluorescein, Pyren, Rhodamin, BODIPY dyes and their analogues;
(b-4) a contrast agent for magnetic resonance imaging (MRI), preferably Gd, Fe, Mn and most preferably Gd in the form of Gd(III) in the form of a chelate complex;
(b-5) an atom or atomic group suitable for imaging by X-ray based technology, preferably iodine or atomic groups containing iodine.
(b-6) an atom or atomic group derived from a therapeutic agent. Said atom or atomic group may have therapeutic activity as such or after cleavage of cyclopeptide-containing moieties to thereby release the underlying therapeutic agent. Preferably, said therapeutic agent is a therapeutic agent suitable for the treatment of cancer or fibrosis.
   If the treatment indication is cancer, the therapeutic agent is preferably selected from alkylating agents, anti-metabolites, anthracyclines, plant alkaloids, topoisomerase inhibitors and other anti-tumor drugs. More specifically, the following can be mentioned: platinum based compounds, antibiotics with anti-cancer activity, anthracyclines, anthracenediones, alkylating agents, antimetabolites, Antimitotic agents, taxanes, taxoids, microtubule inhibitors, Vinca alkaloids, folate antagonists, topoisomerase inhibitors, antiestrogens, antiandrogens, aromatase inhibitors, GnRh analogs, inhibitors of 5α-reductase, bisphosphonates, a metabolic inhibitor, preferably a mTOR inhibitor; an epigenetic inhibitor, preferably a DNMT inhibitor; an anthracycline antibiotic; a camptotheca; an anthracycline; histone deacetylase (HDAC) inhibitors, proteasome inhibitors, JAK2 inhibitors, tyrosine kinase inhibitors (TKIs), PI3K inhibitors, Protein kinase inhibitors, Inhibitors of serine/threonine kinases, inhibitors of intracellular signaling, inhibitors of Ras/Raf signaling, MEK inhibitors, AKT inhibitors, inhibitors of survival signaling proteins, cyclin dependent kinase inhibitors, therapeutic monoclonal antibodies, TRAIL pathway agonists, anti-angiogenic agents, metalloproteinase inhibitors, cathepsin inhibitors, inhibitors of urokinase plasminogen activator receptor function, immunoconjugates, antibody drug conjugates, antibody fragments, bispecific antibodies, bispecific T cell engagers (BiTEs). Said anticancer drug is preferably selected from the group consisting of 5-fluorouracil, cisplatin, irinotecan hydrochloride, epirubicin, paclitaxel, docetaxel, camptothecin, doxorubicin, rapamycin, 5-azacytidine, doxorubicin irinotecan, topotecan (type 1 topoisomerase inhibitors), amsacrine, etoposide, etoposide phosphate and teniposide (topoisomerase-type 2 inhibitors); UFT, capecitabine, CPT-II, oxaliplatin, cyclophosphamide, methotrexate, navelbine, epirubicin, mitoxantrone, raloxifen, mitomycin, carboplatinum, gemcitabine, etoposide and topotecan.
   Further suitable therapeutic agents for the treatment of cancer are disclosed, for instance, in “Cancer Drugs” by Judith Matray-Devoti, Chelsea House, 2006; “Physicians' Cancer Chemotherapy Drug Manual 2015” by Edward Chu, Vincent T DeVita, Jr., Jones & Bartlett Learning 2015; “Cancer Chemotherapy and Biotherapy: Principles and Practice” by Bruce A. Chabner, Dan L. Longo, Wolters Kluwer, 2011; “Drugs in Cancer Care” by Rachel Midgley, Mark R. Middleton, Andrew Dickman, David Kerr (Eds.), Oxford University Press 2013. The drugs disclosed in these books can be used as therapeutic agents when practicing the present invention. The disclosures of therapeutic drugs in these references is therefore incorporated herein.
   If the treatment indication is fibrosis, the therapeutic agent is preferably selected from therapeutic drugs suitable for the treatment of fibrosis. Such therapeutic drugs are disclosed, for instance, in “Cystic Fibrosis in the 21st Century” by Andrew Bush (Ed.), S. Karger, 2006; “Liver Fibrosis: New Insights for the Healthcare Professional: 2013 Edition” by Q. Ahton Acton, ScholarlyEditions, 2013; “Idiopathic Pulmonary Fibrosis: A Comprehensive Clinical Guide” by Keith C. Meyer, Steven D. Nathan, Springer, 2014; “New Insights into the Pathogenesis and Treatment of Idiopathic Pulmonary Fibrosis: A Potential Role for Stem Cells in the Lung Parenchyma and Implications for Therapy” by M. Gharaee-Kermani et al. in Pharmaceutical Research, 2007, 24, 819-841; “Pulmonary Fibrosis: pathogenesis, etiology and regulation” by M. S. Wilson and T. A. Wynn in Mucosal Immunol. 2009, 2, 103-121. Specific preferred therapeutic drugs are preferably selected from the drugs and drug classes disclosed listed in Table II of the review article by Gharaee-Kermani et al. cited above.
   If the treatment indication is a Covid-19 infection, the therapeutic agent may be any agent having experimentally established or suspected activity in the treatment of such infections, irrespective whether they are already used in clinical practice or are still in development stage. Agents currently used for or in development for the treatment of Covid-19 infections are for instance antiviral agents, including for example the anti-ebola agent remdesivir or the anti-influenza agent favilavir, kinase inhibitors such as ATR-002, anti-inflammatory agents including glucocorticoids, antagonists of IL-1 or IL-6 such as anakinra and tocilizumab, respectively, anti-infective agents such as ivermectin or agents for the treatment of other lung conditions like fibrosis. Reference can thus be made to the literature and agents mentioned above for fibrosis.

The active atom or group of atoms can be bonded to the cyclopeptide (or cyclopeptides) via atomic groups acting as a spacer. The atomic group acting as a spacer is typically a linear chain of 2 to 20 and preferably 3 to 10 atoms selected from C, N, O, P and S, preferably alkylene groups, which optionally carry one or more substituents, the remaining valences being saturated by hydrogen. This linear chain may be interrupted by one or more cyclic structures preferably such having 5 ring atoms, and more preferably a triazole ring. Bonding to the amino group of the side chain of N(Me)K is typically accomplished by means of an amide bond. Bonding of the spacer to the active atom or atomic group Aa′ in formula (1a′), (1b), (1c) or (1d) can also be accomplished by means of an amide bond but a direct covalent bond is also possible.

The atomic group acting as a spacer, for instance in the above formulae (Ia) to (If), is further described hereinbelow. It may in one embodiment be characterized by the following formula (IIIa):

*—C(O)—(CH₂)ₖ-(taz)ₗ-(CH₂)ₘ₋ (IIIa)

wherein taz stands for a triazole ring with all three nitrogen atoms being adjacent to each other, 1 may be 0 or 1 and each of k and m is an integer selected from 0 to 20 such that k+m=2-20. The asterisk (*) marks the point of attachment of the cyclopeptide.

In another embodiment, additional divalent functional groups may be present, as shown by the following formulae (IIIb) to (IIIf):

*—C(O)—(CH₂)ₖ—NH—CO—(CH₂)ₘ₋ (IIIb)

*—C(O)—(CH₂)ₖ—CO—NH—(CH₂)ₘ₋ (IIIc)

*—C(O)—(CH₂)ₖ-(taz)ₗ-(CH₂)ₒ—CO—NH—(CH₂)ₘ₋ (IIId)

*—C(O)—(CH₂)ₖ-(taz)ₗ-(CH₂)ₒ—NH—CO—(CH₂)ₘ₋ (IIIe)

*—C(O)—(CH₂)ₖ—CO—NH—(CH₂)ₒ-(taz)ₗ-(CH₂)ₘ₋ (IIIf)

*—C(O)—(CH₂)ₖ—NH—CO—(CH₂)ₒ-(taz)ₗ-(CH₂)ₘ₋ (IIIf)

wherein taz and 1 have the same meanings as indicated above with respect to formula (IIIa). k, m and, if present, o are integers independently selected from the range of 0 to 20 such that k+m=2-20 and k+m+o=2-20, respectively. The asterisk (*) marks again the point of attachment of the cyclopeptide.

According to one embodiment, one or more of the spacers may carry one or more independently selected substituents. Each of these substituents is not particularly restricted. According to a preferred embodiment, the substituent is itself a moiety containing a spacer and a cyclopeptide, preferably a spacer S and a cyclopeptide Cp as described herein. It is even possible that the spacer part of said substituents is further substituted to form a dendrimeric structure, which may have up to 3 generations of substituents attached to the 0 generation spacers depicted in formulae (Ia) to (Ie).

In other embodiments, especially in connection with active atoms or active atomic groups that are suitable for therapeutic purposes, the spacer may be cleavable under physiological conditions. Such cleavable spacers are not particularly limited and may be selected from the spacers described in WO 2009/117531 A, WO 2015/123679 A, Younes et al. N. Engl. J. Med. 2010; 363:1812-1821; Dorywalska et al. Mol. Cancer Ther. 2016; 15(5):958-970, Jain et al., Pharm. Res. 2015; 32(11):3526-3540, and references cited therein.

If the active atom is a metal ion, bonding is typically accomplished via a chelating group, for example, as described in Chem. Soc. Rev. 2011; 40:3019-3049.⁴⁰ Binding of the metal ion by the chelating group preferably occurs via complex bonds (Lewis acid/base interactions) effected by the N and O atoms of the chelating group. The chelating group is however not particularly limited as long as it is capable of forming a chelate complex with the metal ion of interest, which is preferably stable under physiological conditions for a time period that is sufficiently long for carrying out the intended diagnostic method. Preferred chelators or chelator-containing functional groups are those mentioned in Chem. Soc. Rev. 2014; 43:260-290 (DOTA, B-DO2A, 3p-C-DEPA, TCMC, Oxo-DO3A, TETA, E2A, CB-TE2A, CB-TE1A1P, CB-TE2P, MM-TE2A, DM-TE2A, Diamsar, NOTA, NETA, and TACN-TM, DTPA, 1B4M-DTPA, CHX-A″-DTPA, AAZTA, DATA, H₂dedpa, H₄octapa, H₂azapa, H₅decapa, BCPA, CP256, YM103, DFO, PCTA, H₆phospha, PCTA, HEHA, PEPA), bispidines (as mentioned in Dalton Trans. 2018; 47: 9202-9220), radiohybrid ligands (as described by Wurzer et al. in J. Nucl. Med. 2019, doi: 10.2967/jnumed.119.234922), hydroxypyridinone ligands (as described in Dalton Trans. 2019; 48:4299-4313 or Bioconjugate Chem. 2015; 26:2579-2591), picolinic acid based chelators (as mentioned in Dalton Trans. 2017; 46:14647-14658, Inorg. Chem. 2016; 55:12544-12558, or Bioconjugate Chem. 2017; 28:2145-2159) and especially, chelating groups who allow for conjugation of more than one peptide without additional branched linkers, such as fusarinine c (as described in J. Label. Compd. Radiopharm. 2015; 58:209-214), DOTPI (as described in Chem. Eur. J. 2013; 19:7748-7757), DOTGA (as described in Chem. Commun. 1998, 1381), NOTGA (as described in Bioconjugate Chem. 2012; 23:2229-2238), NODAPA (as described in Bioorg. Med. Chem. Lett. 2008; 18:5364-5367), DOTAZA (as described in Chem. Asian J. 2014; 9:2197-2204), HBED-CC (as described in Eur. J. Nucl. Med. 1986; 12.397-404), HBED-NN (as described in J. Org. Chem. 2019; 84:7501-7508), (NH₂)₂sar (as described in Inorg. Chem. 2011; 50: 6701-6710) or TRAP (as mentioned for instance in Dalton Trans. 2015; 44:11137). Particularly preferred are TRAP, its tetravalent homologue DOTPI, DOTAZA, and analogues and derivatives of these chelating groups. Typical structures of these chelating groups are represented by formulae (IVa) to (IVd) below:

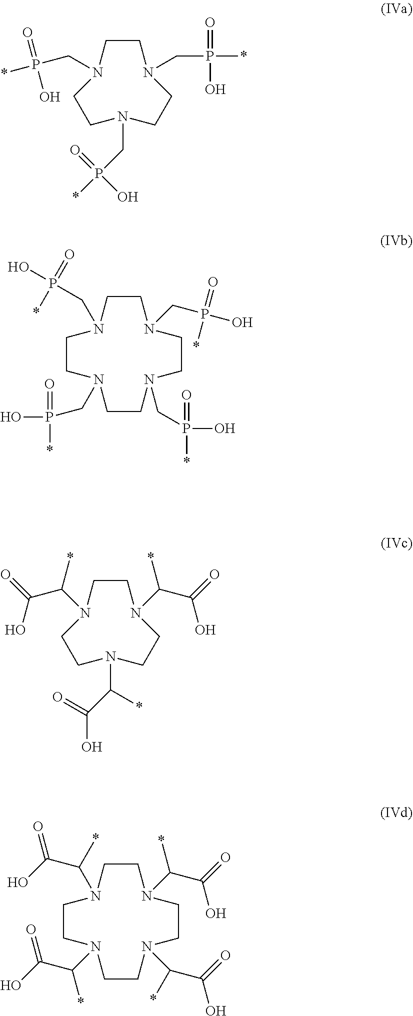 [SMILES: *P(=O)(O)CN1CCN(CP(*)(=O)O)CCN(CP(*)(=O)O)CC1 ; *P(=O)(O)CN1CCN(CP(*)(=O)O)CCN(CP(*)(=O)O)CCN(CP(*)(=O)O)CC1 ; *C(C(=O)O)N1CCN(C(*)C(=O)O)CCN(C(*)C(=O)O)CC1 ; *C(C(=O)O)N1CCN(C(*)C(=O)O)CCN(C(*)C(=O)O)CCN(C(*)C(=O)O)CC1]

wherein the asterisk (*) marks the point of attachment of the atomic group acting as a spacer. If the number of cyclopeptides and associated spacers (as characterized by variable n) is less than the number of valences of the chelating group, the remaining valences shown by the asterisk are saturated by hydrogen or another atomic group, preferably a group selected from —CH₂—COOH and —CH₂—CH₂—COOH.

### Manufacture of the Conjugates of the Invention

The conjugates of the invention may be synthesized using standard materials and methods known in the art. If the conjugate is a chelate, the formation of the chelate is typically performed as the last step. That is, a suitable procedure includes one or more steps for forming a precursor, as described below, followed by reaction of the precursor with the atom, atomic group or ion to be chelated. Said final reaction is typically conducted under usual conditions for reactions of this kind which are known to the skilled artisan. In a preferred setting, the reaction is conducted at ambient temperature (room temperature, e.g. 20-25° C.). Also preferred, the reaction is conducted at temperatures ranging from ambient temperature (room temperature) to 37° C.

Said ion may be provided in the form of a salt, wherein the salt-forming counter-ion may be selected from the group consisting of sulfates, fluorides, chlorides, bromides, nitrates, phosphates, carbonates, hydrogencarbonates, sulfonates, acetates, and mixtures thereof. In a further preferred embodiment, the ion is provided in the form of a solution.

The precursor is preferably prepared using a modular approach based on Click chemistry to link the chelating group (or central moiety) to the cyclopeptide moiety/moieties. The spacer/spacers is/are formed in situ during said coupling reaction. The starting materials contain themselves precursors of the spacers with functional groups suitable for Click chemistry couplings at their termini.

Cyclopeptides carrying precursors of spacers at their (NMe)K residue may be obtained by reacting the respective precursor, which carries a carboxyl group at the cyclopeptide-binding terminus and which may be activated using for instance HATU, HOBt and DIPEA, is reacted with the respective cyclopeptide under standard amide coupling conditions for instance as described in Maltsev O V, et al., Angew. Chem. Int. Ed. 2016; 55:1535-1539 and/or WO 2017/046416 A1.

The cyclopeptide can be synthesized by applying suitably adapted materials and procedures described in the literature, for instance in Maltsev O V, et al., Angew. Chem. Int. Ed. 2016; 55:1535-1539 and/or WO 2017/046416 A1.

### Specific Conjugates of the Invention

In the following, specific conjugates of the invention are shown. Conjugates of the invention include both the conjugates as shown below as well as the corresponding conjugates obtainable by incorporating a non-radioactive metal ion or a radionuclide such as ⁶⁸Ga into the structures shown below.

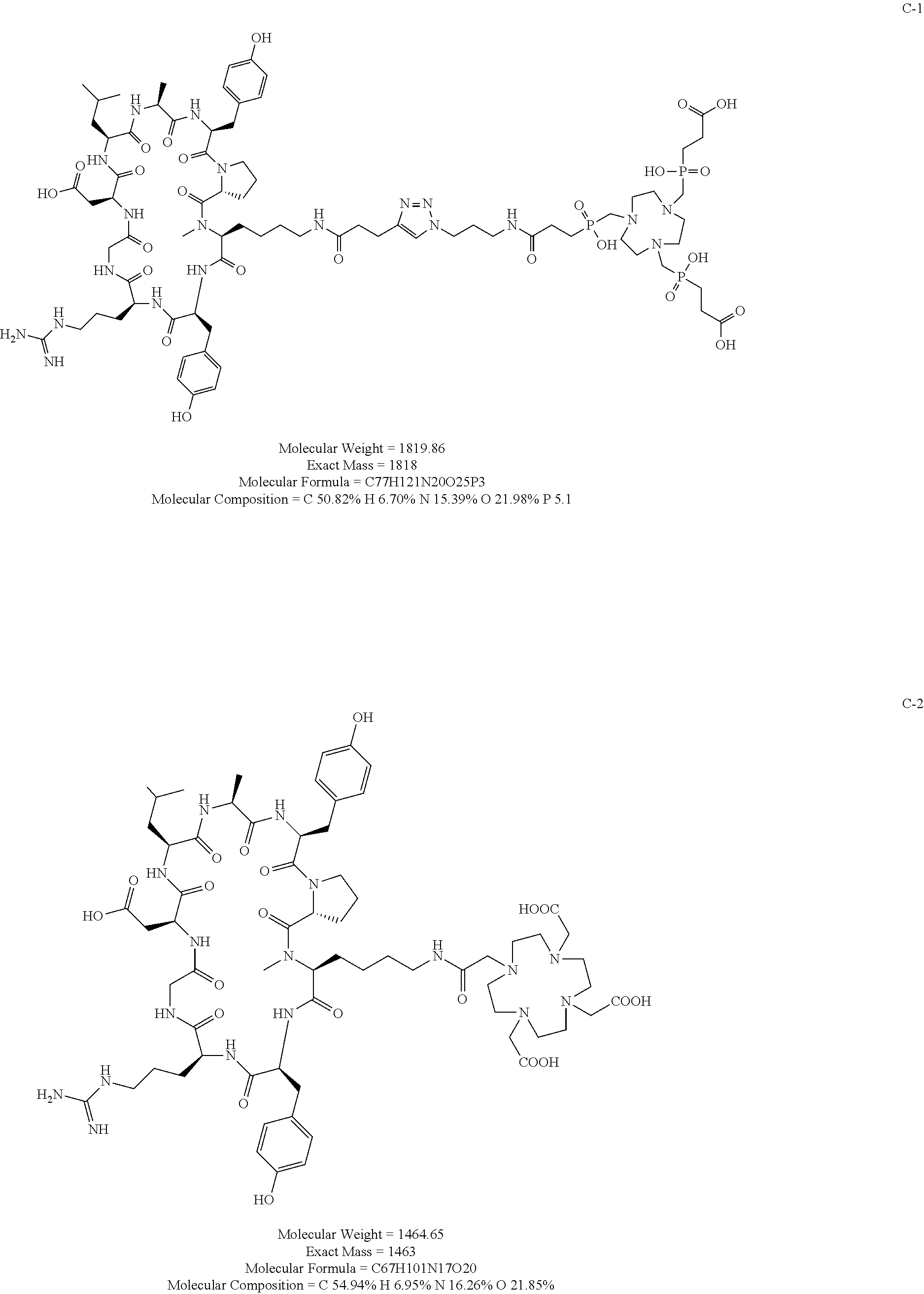 [SMILES: CC(C)C[C@@H]1NC(=O)[C@H](CC(=O)O)NC(=O)CNC(=O)[C@H](CCCNC(=N)N)NC(=O)[C@H](Cc2ccc(O)cc2)NC(=O)[C@H](CCCCNC(=O)CCc2cn(CCCNC(=O)CCP(=O)(O)CN3CCN(CP(=O)(O)CCC(=O)O)CCN(CP(=O)(O)CCC(=O)O)CC3)nn2)N(C)C(=O)[C@H]2CCCN2C(=O)[C@H](Cc2ccc(O)cc2)NC(=O)[C@H](C)NC1=O ; CC(C)C[C@@H]1NC(=O)[C@H](CC(=O)O)NC(=O)CNC(=O)[C@H](CCCNC(=N)N)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H]2CCCN2C(=O)[C@H](Cc2ccc(O)cc2)NC(=O)[C@H](C)NC1=O]

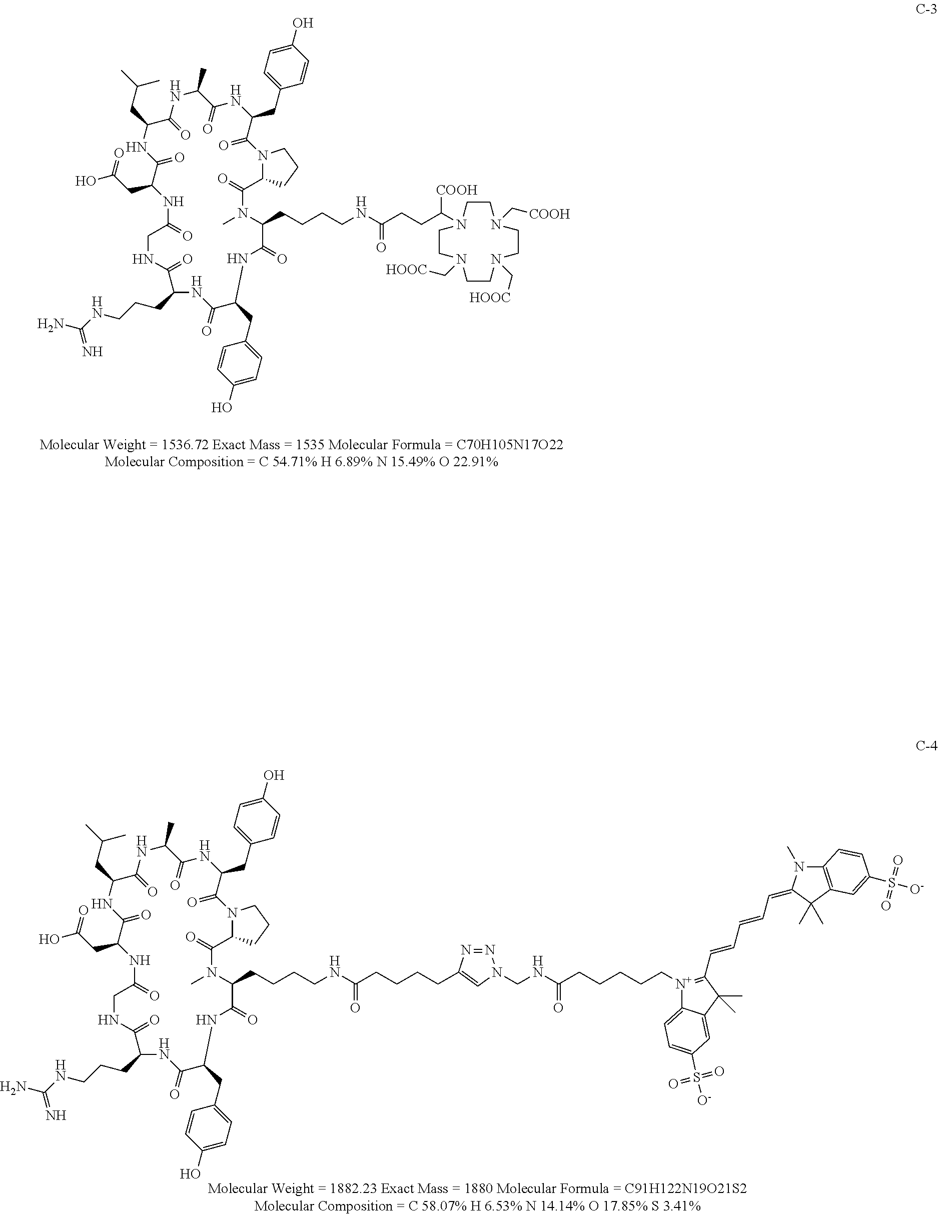

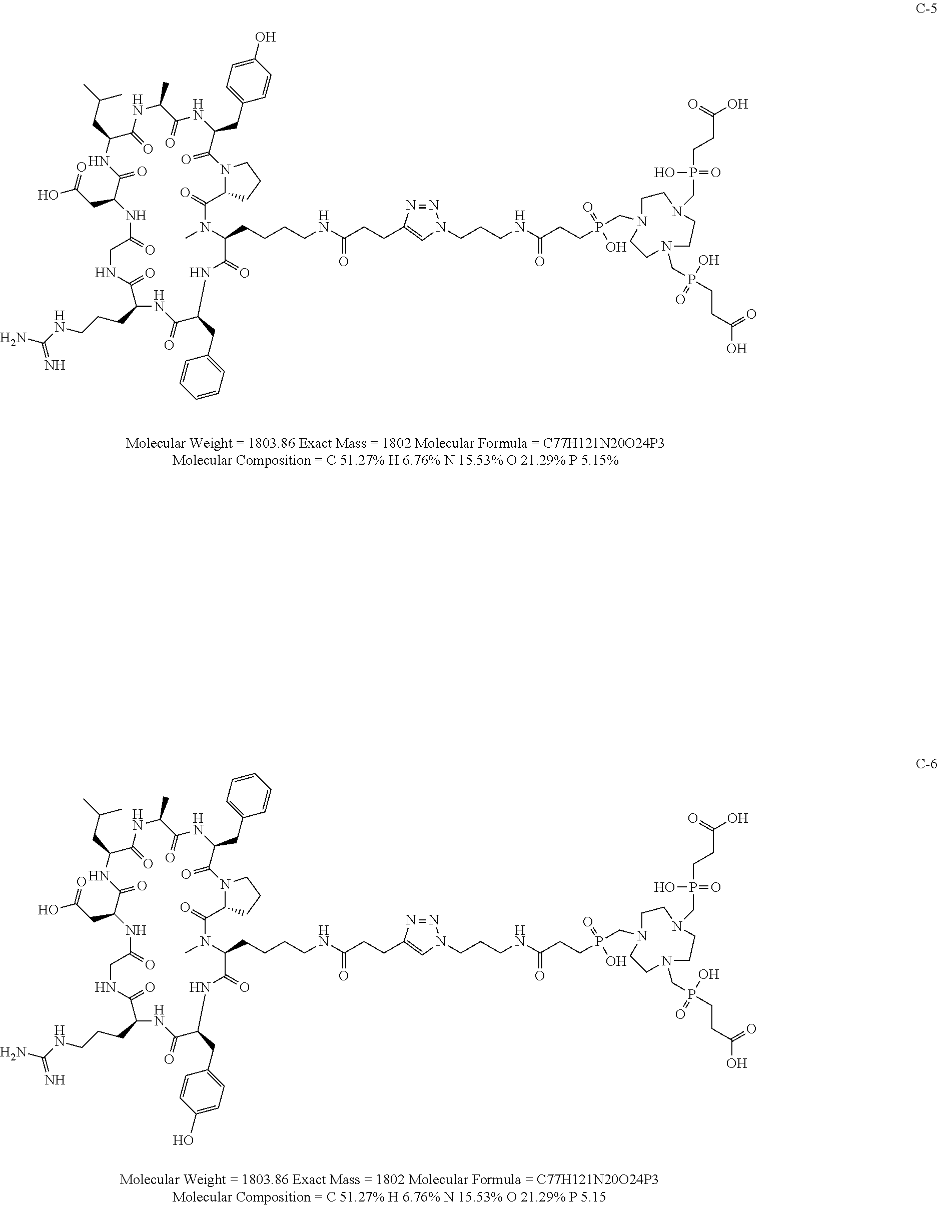

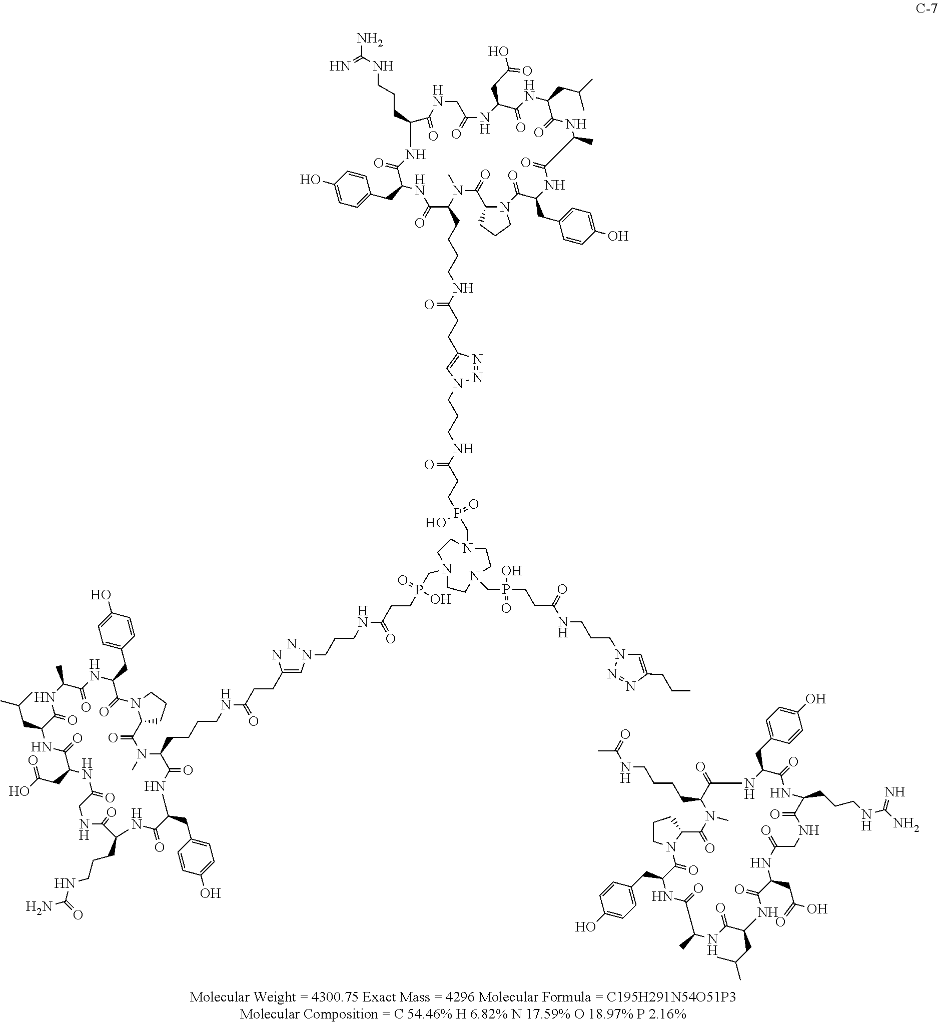

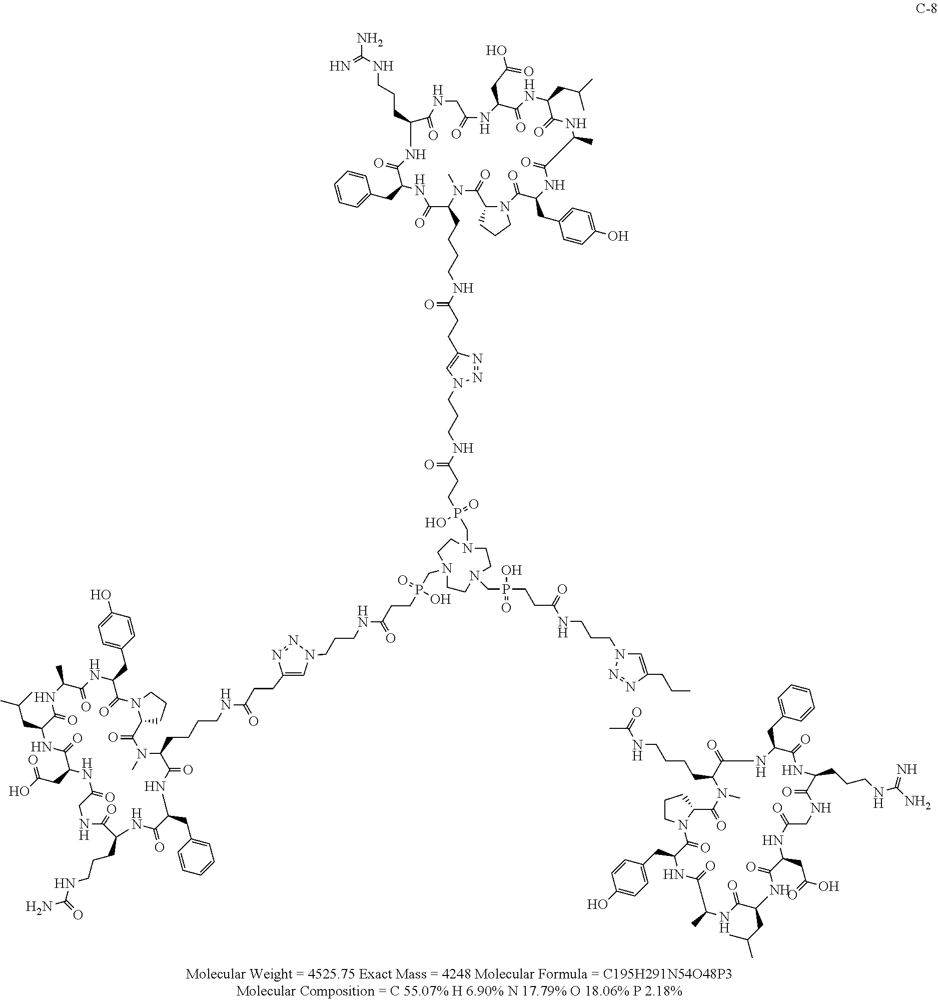

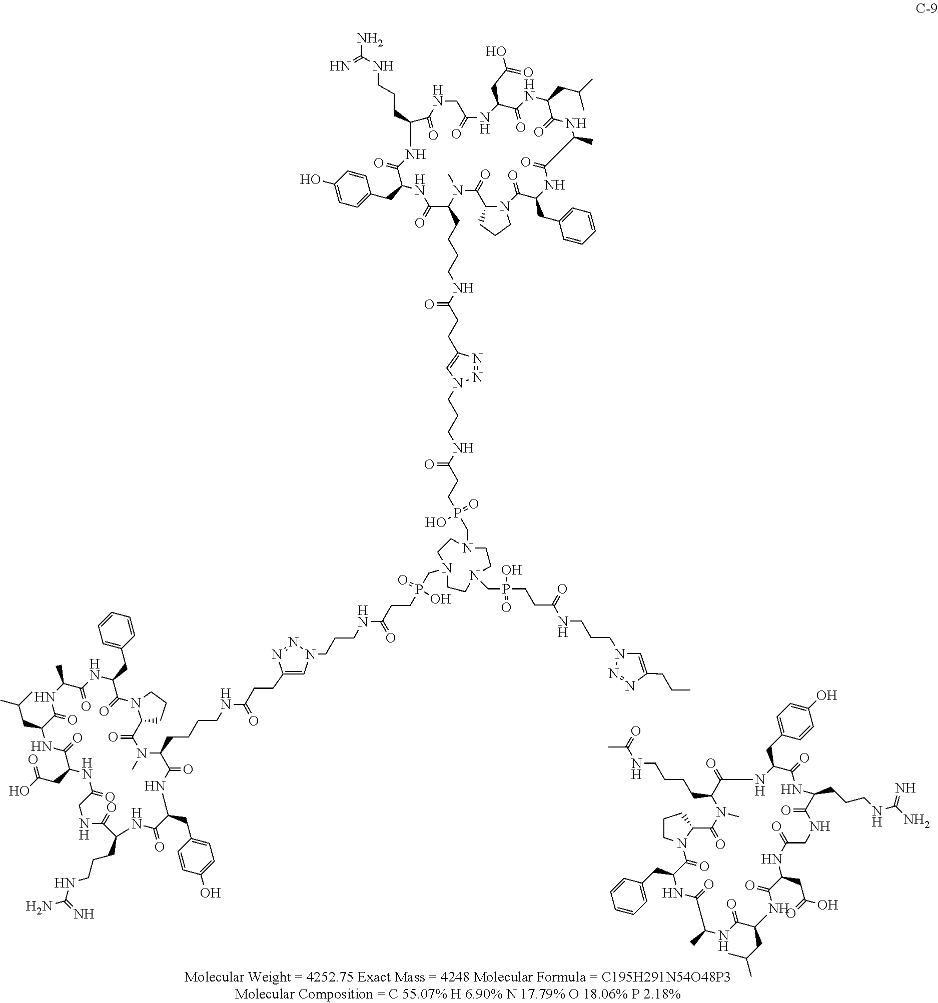

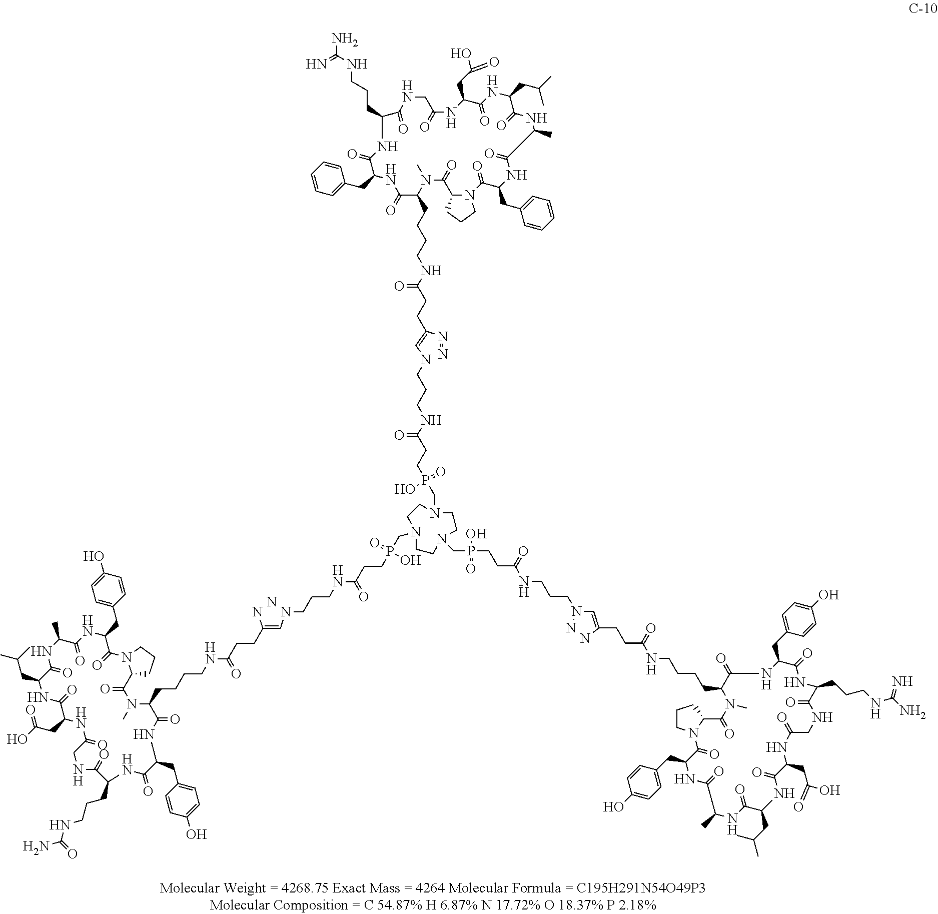

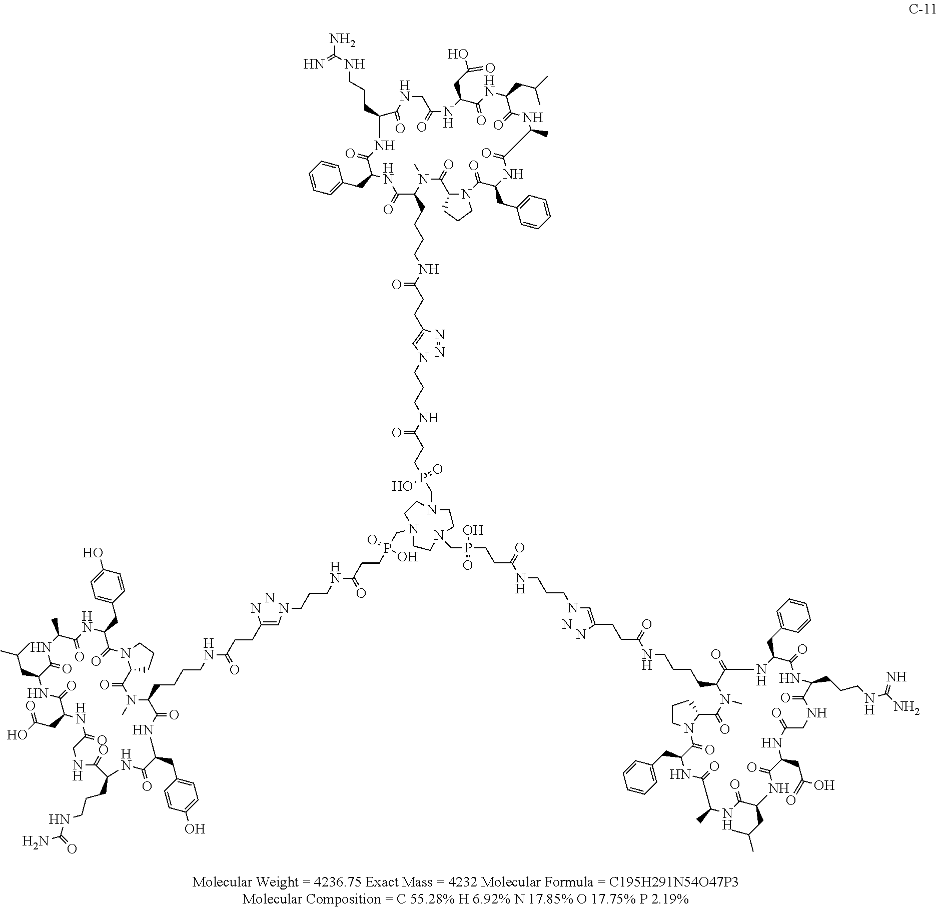

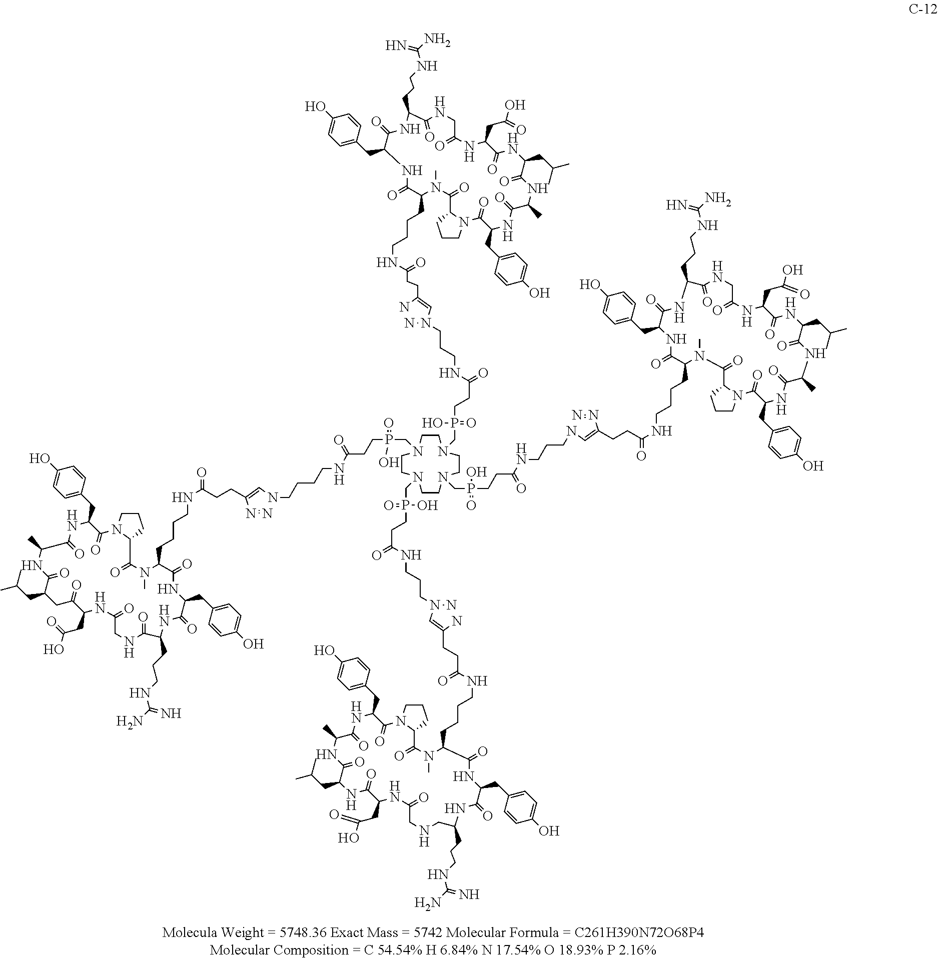

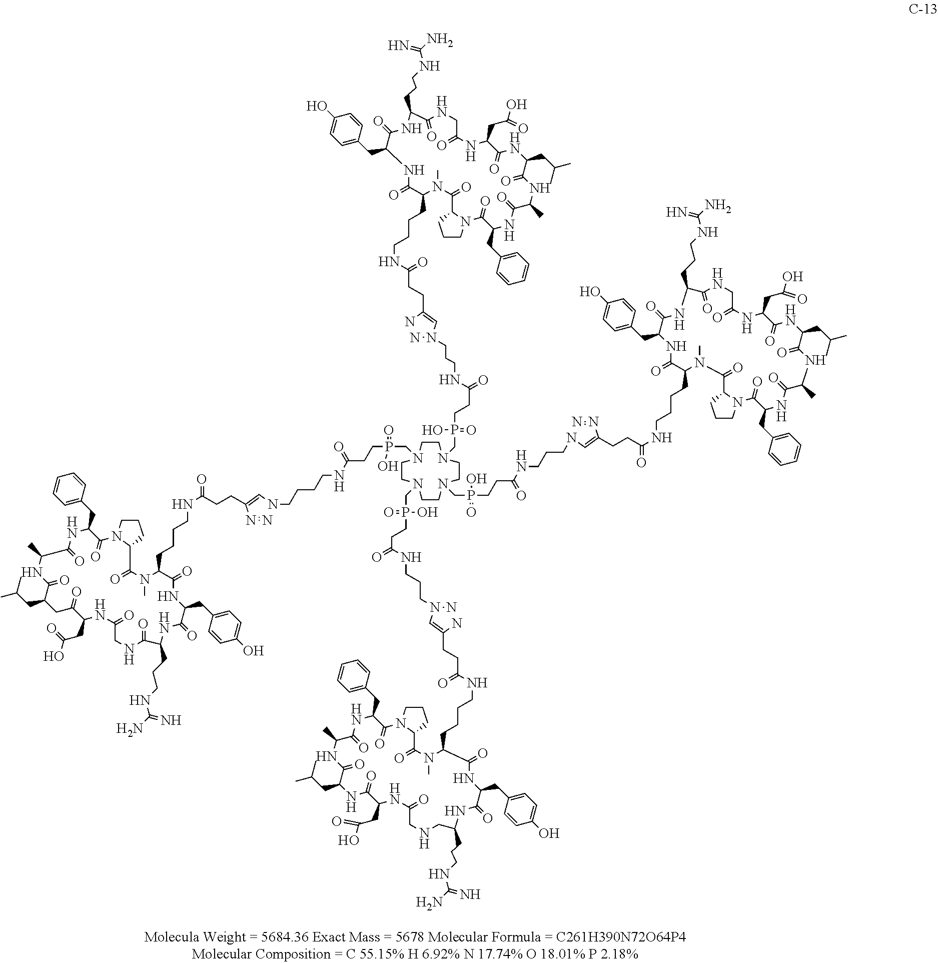

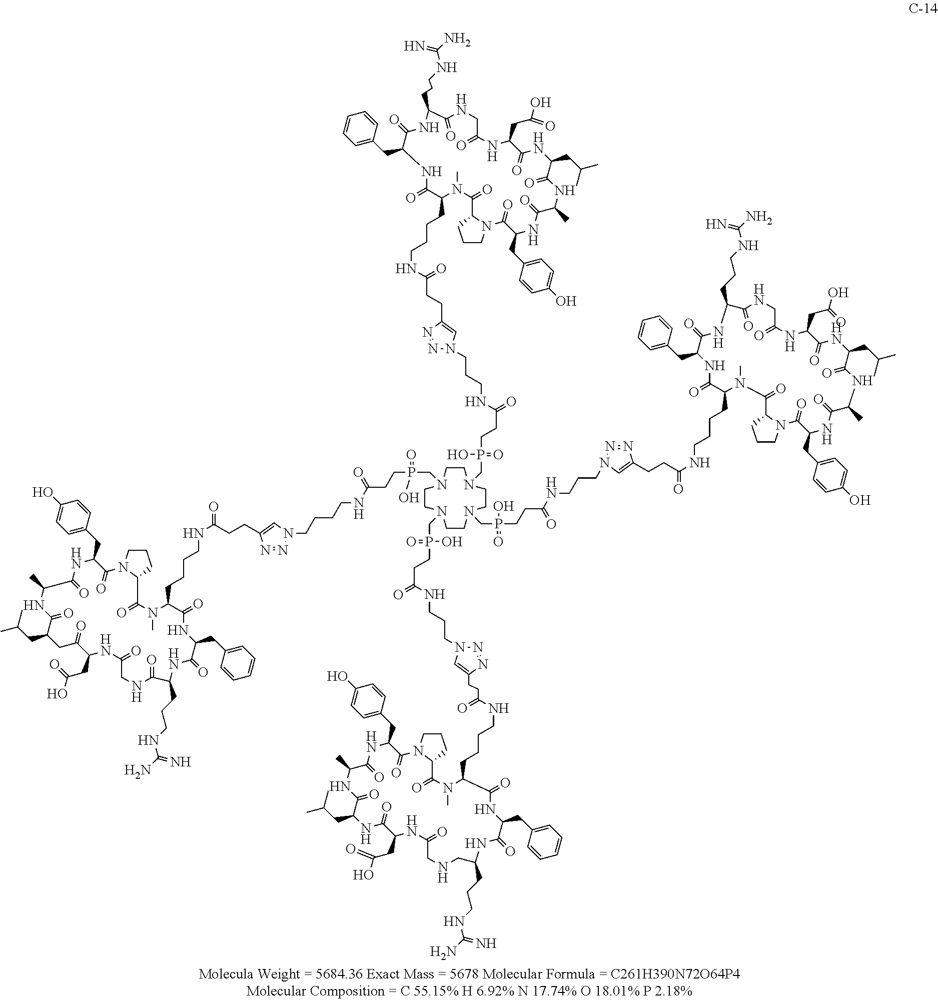

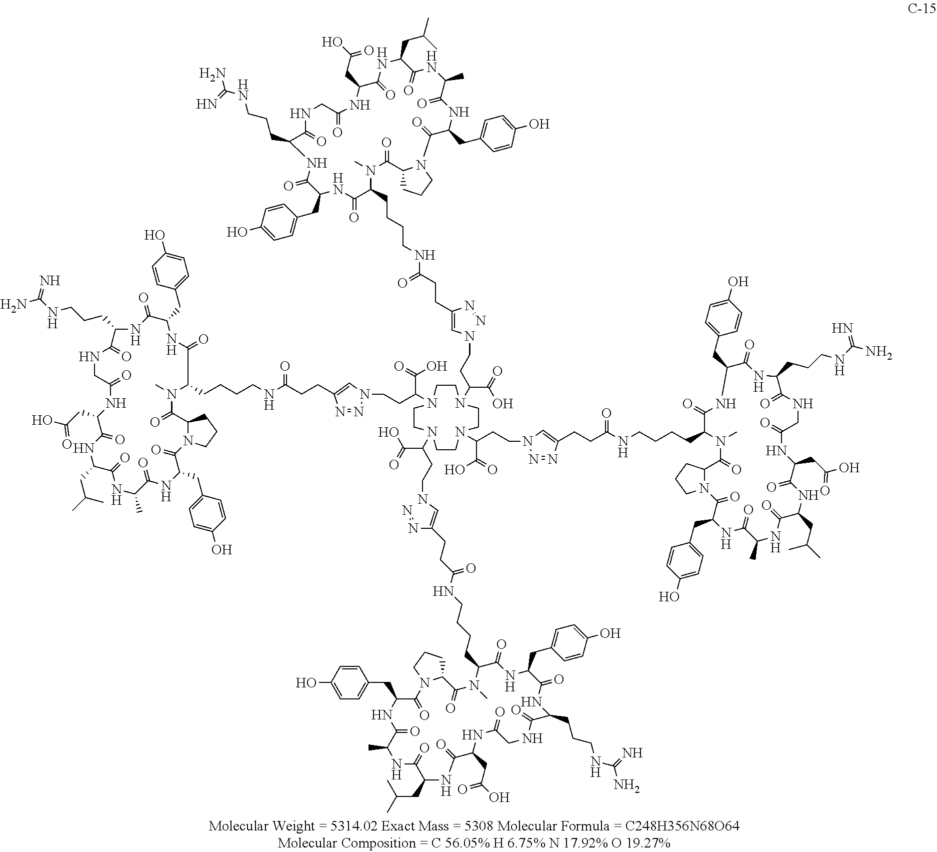

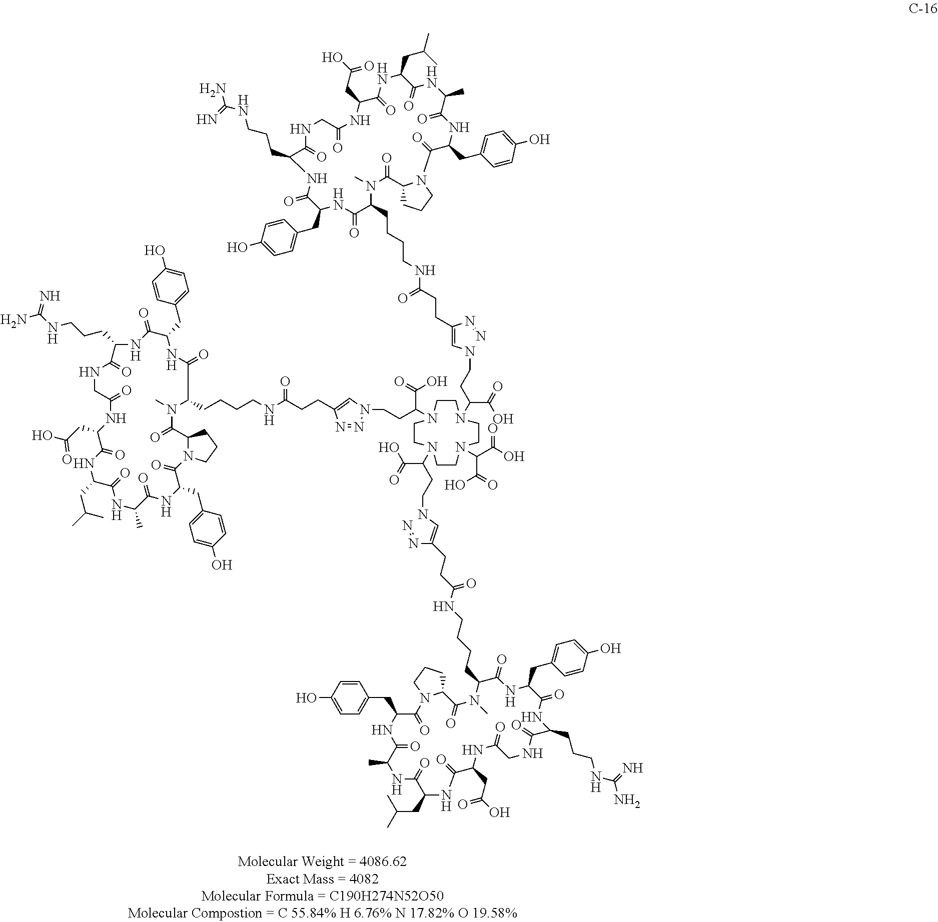

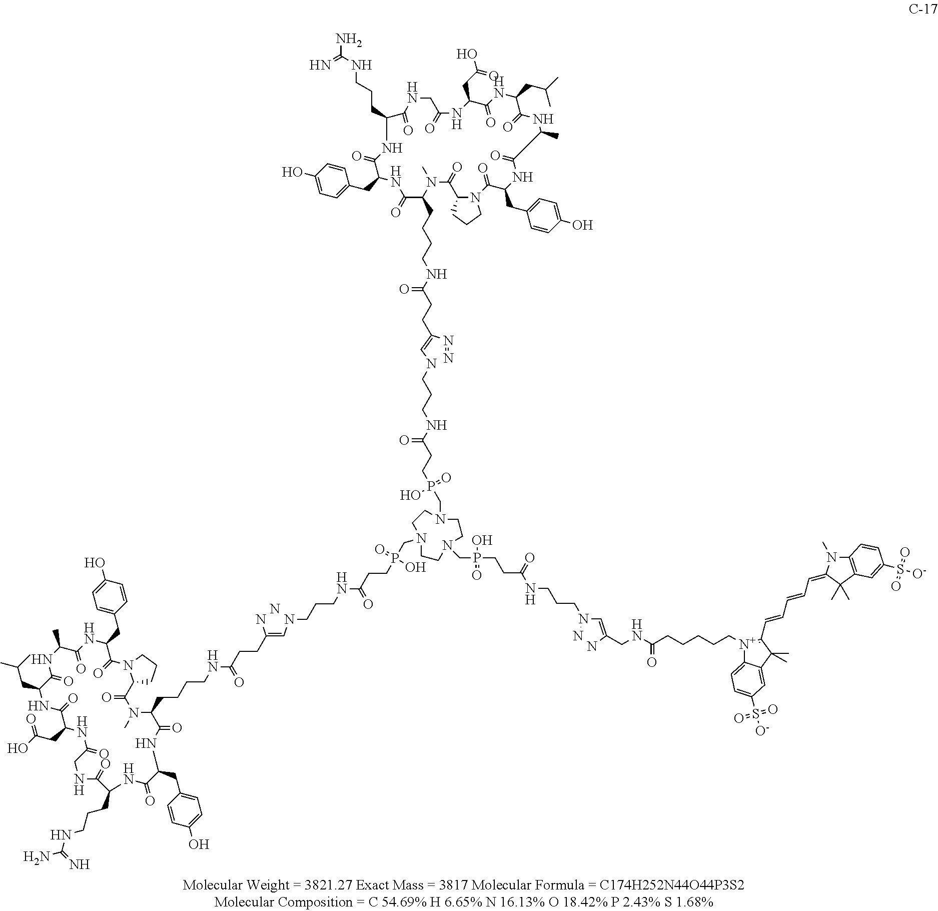

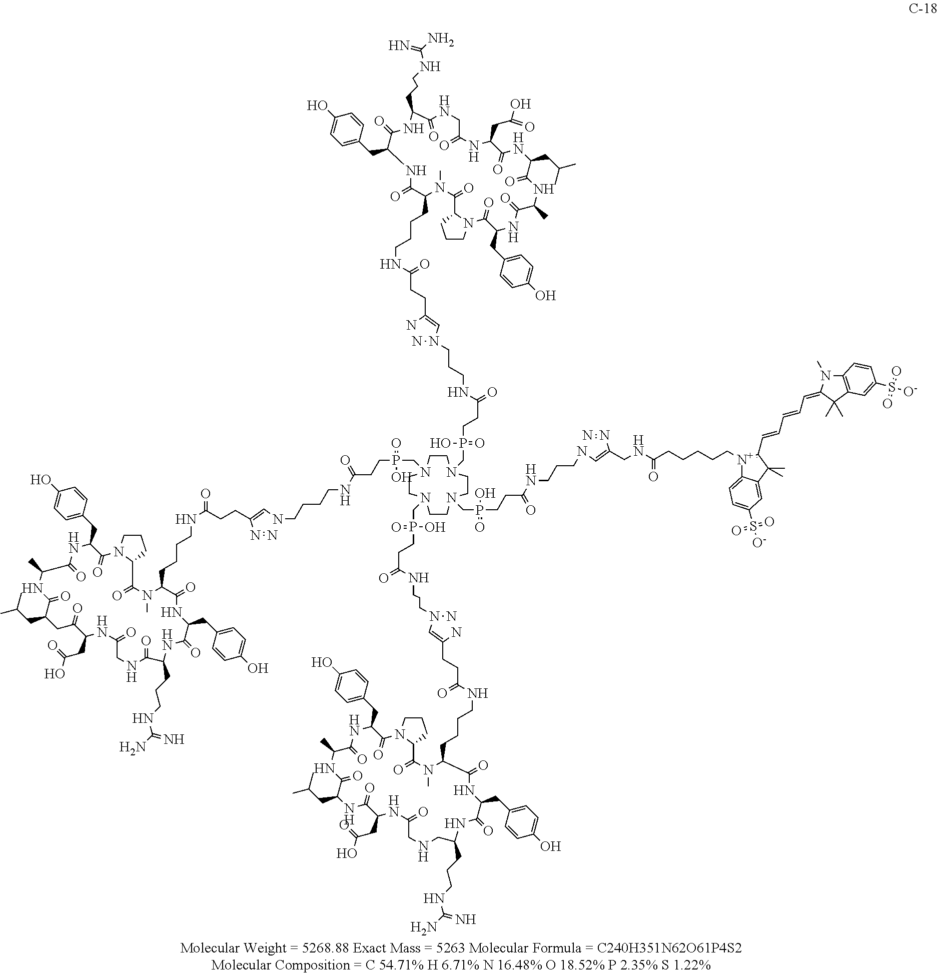

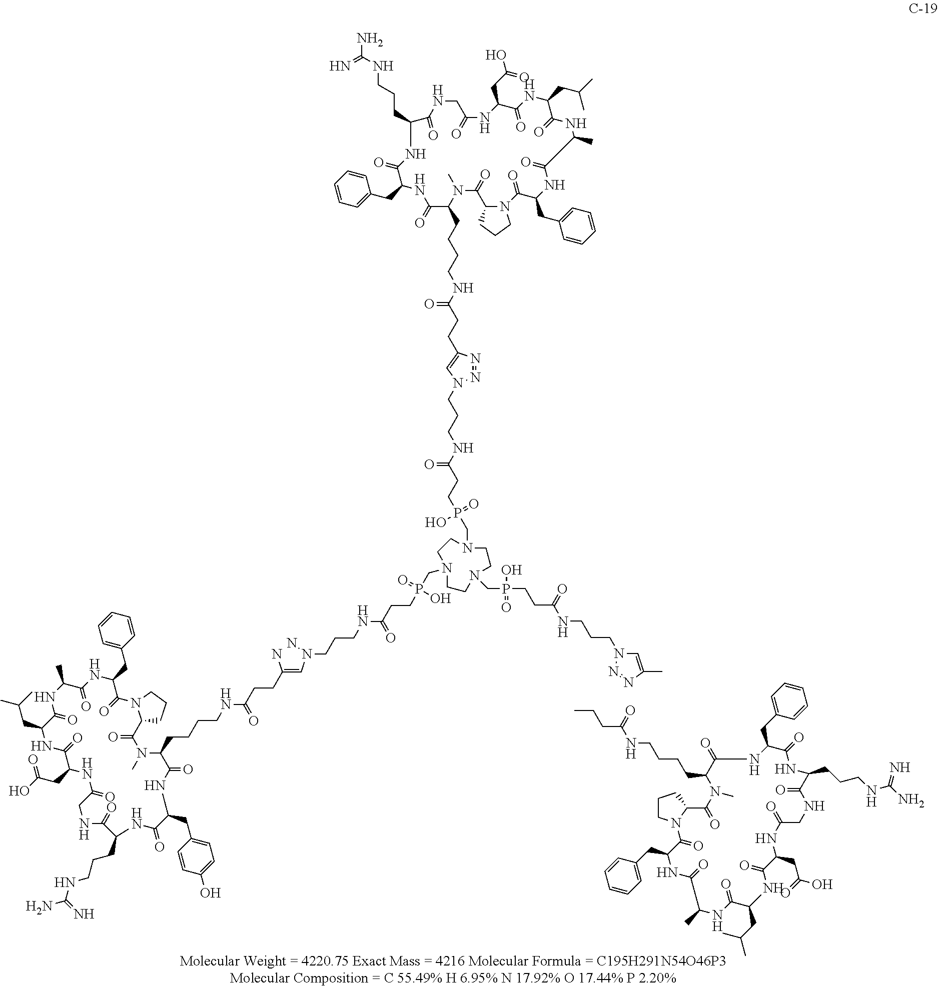

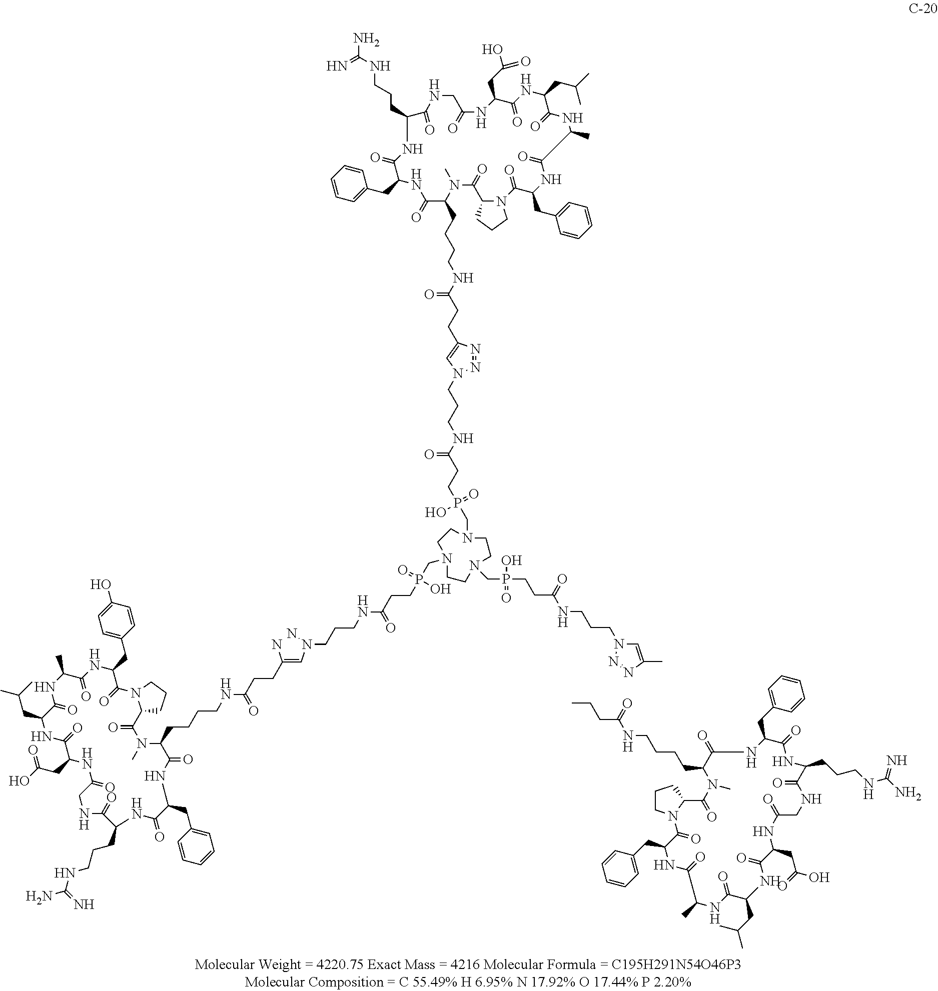

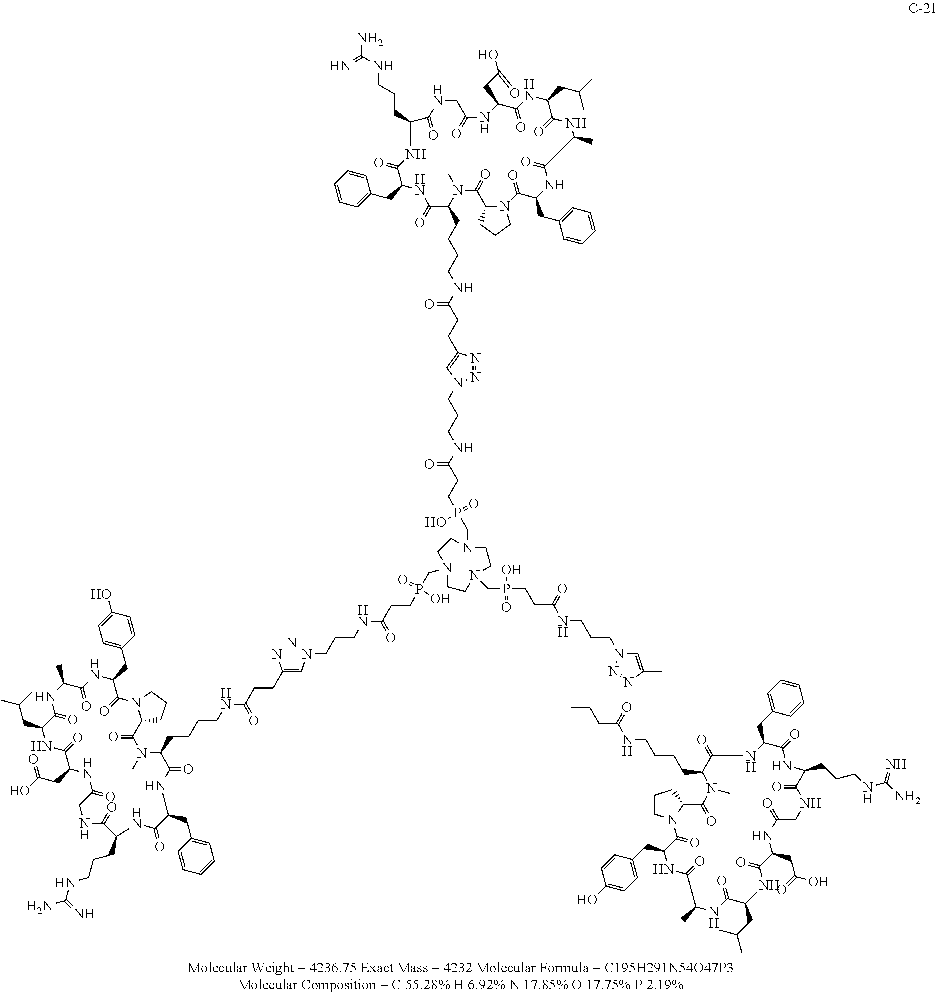

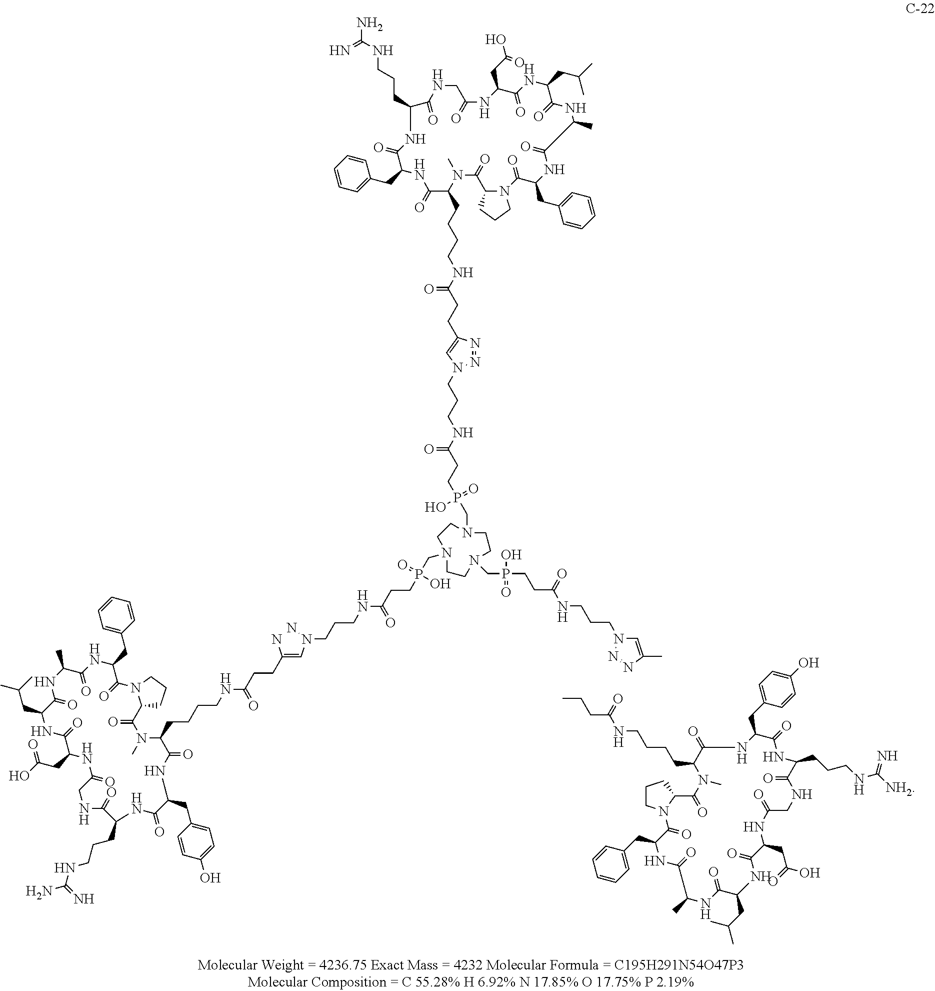

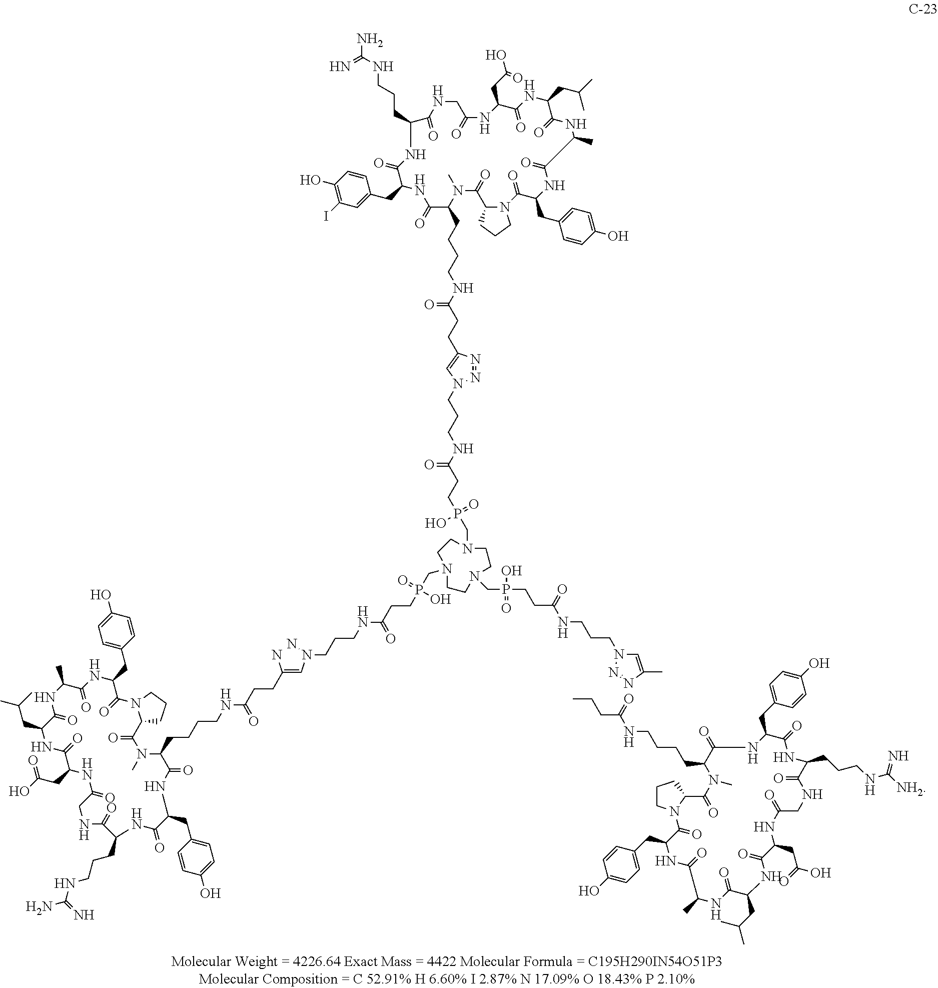

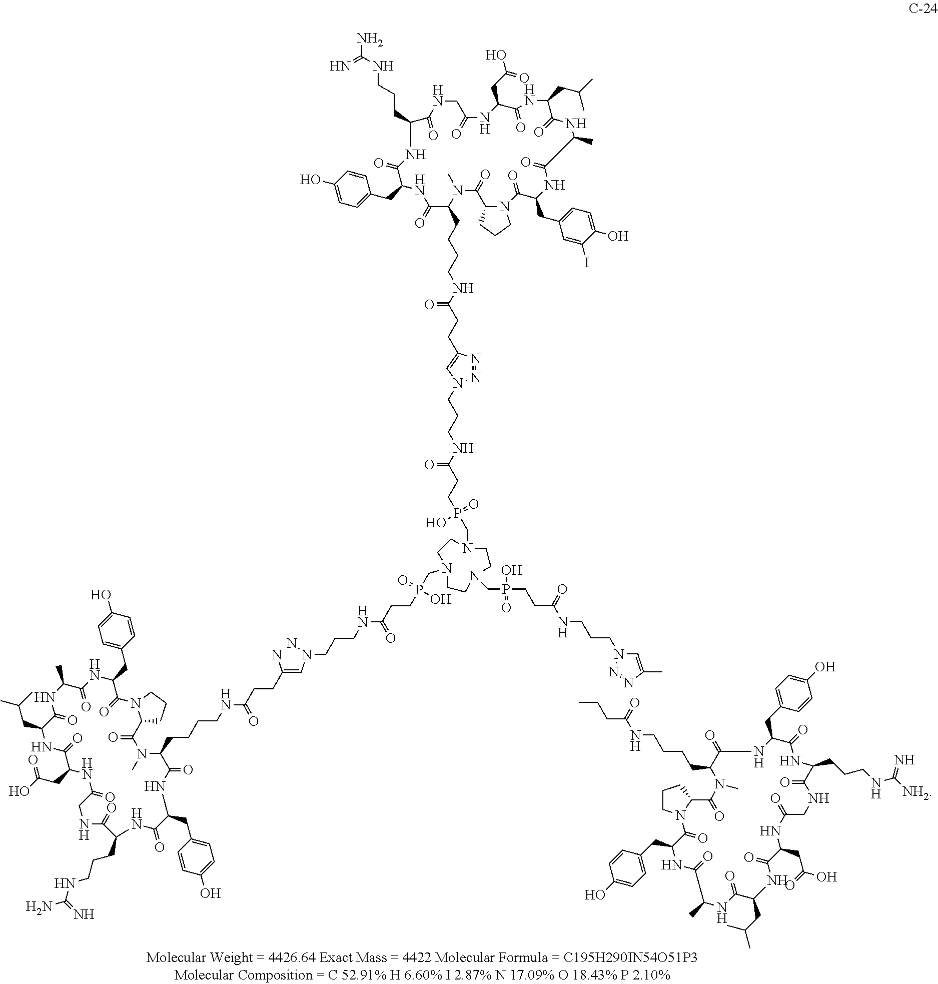

### Building Blocks of the Invention

The present invention further relates to building blocks that can be used for obtaining the conjugates of the present invention.

A first type of building block of the present invention is the group of compounds corresponding to the chelate complexes described above, but without the coordinated atom (such as Ga-68). These building blocks of the present invention may be characterized by the following formula (IIa):

Cg(SCp)ₙ (IIa)

wherein Cg stands for a chelating group, S stands for an atomic group acting as a spacer, each Cp is a cyclopeptide independently selected from Tyr₂, YRGD and FRGD and n is an integer of from 1 to 4. All of the further information provided above for the corresponding coordinated complexes applies in an analogous fashion to the building blocks of formula (IIa).

The present invention further relates to building blocks, which are modified cyclopeptides that can be used at earlier stages of the synthetic procedure for synthesizing the conjugates and above-mentioned building blocks of the present invention by the convenient Click chemistry. Such building blocks comprise a cyclopeptide moiety selected from Tyr₂, YRGD and FRGD, a functional group that may participate in a Click reaction (e.g. as specified, for instance, in the Wikipedia entry “Click chemistry” in the version of Jan. 24, 2020) and an atomic group linking the cyclopeptide to the functional group via the terminal amino group of the sidechain of the NMe-K residue.

These building blocks of the invention may be represented by the following formula (V):

Cp-L-Fg (V)

wherein Cp represents the cyclopeptide selected from Tyr₂, YRGD and FRGD, L represents the linking group and Fg represents the functional group for carrying out the Click reaction.

The functional group is preferably an azide group, an alkyne group including especially a terminal ethyne group, a dibenzylcyclooctyne group, a trans-cyclooctene group, a tetrazine group, a dibenzocyclooctyne group or a bicyclo[6.1.0]nonyne group.

The linking group typically includes a carbonyl group forming an amide bond with the amino group of the sidechain of the NMe-K residue. It further includes a group of 1 to 15 atoms selected from C, N, O, forming a linear chain between the amide bond and the functional group, which is optionally substituted by one or more substituents, the remaining valences of the chain-forming atoms being saturated by hydrogen atoms. Preferably, said group is an alkylene group with 1 to 15, more preferably 2 to 6, methylene groups.

The following formula BB-1 illustrates this concept for a building block of the invention wherein the Tyr₂ cyclopeptide is linked to an azide functional group via a C₄-alkylene group.

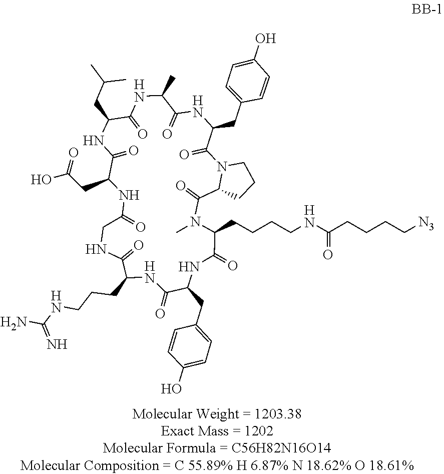 [SMILES: CC(C)C[C@@H]1NC(=O)[C@H](CC(=O)O)NC(=O)CNC(=O)[C@H](CCCNC(=N)N)NC(=O)[C@H](Cc2ccc(O)cc2)NC(=O)[C@H](CCCCNC(=O)CCCCN=[N+]=[N-])N(C)C(=O)[C@H]2CCCN2C(=O)[C@H](Cc2ccc(O)cc2)NC(=O)[C@H](C)NC1=O]

Further useful building blocks are depicted by the following formulae BB-2 to BB-7.

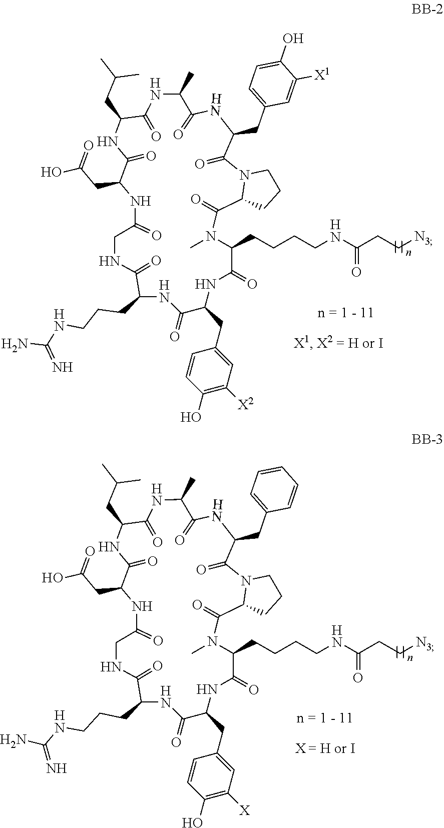

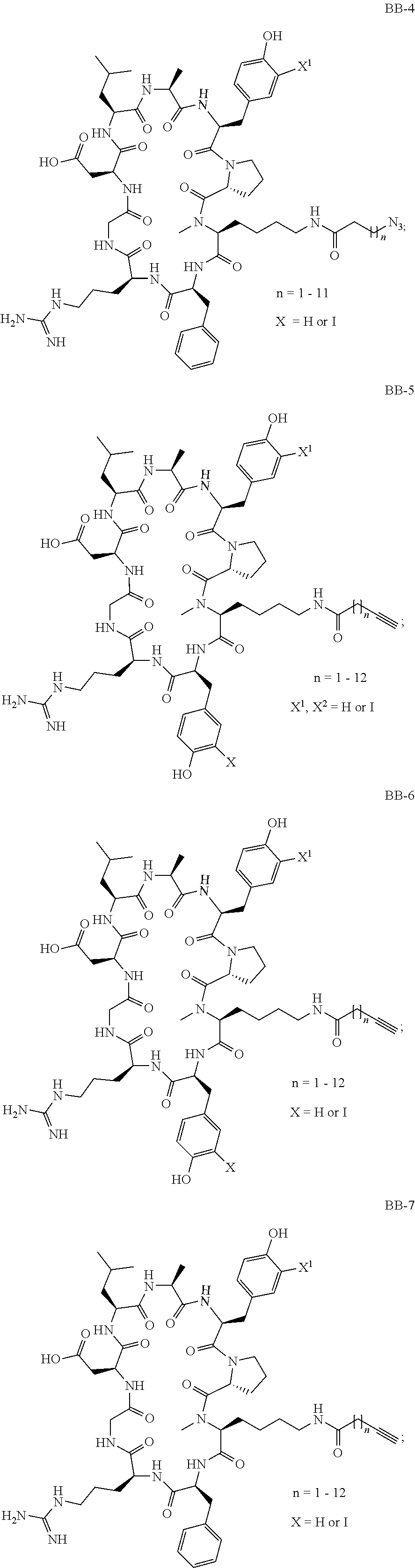

BB-5a refers to the structure BB-5, wherein X¹ and X² are Hydrogen, and n=2.

BB-6a refers to the structure BB-6, wherein X is Hydrogen, and n=2.

BB-7a refers to the structure BB-7, wherein X is Hydrogen, and n=2.

The Tyr₂ cyclopeptide itself is novel and represents another building block of the present invention for obtaining the conjugates of the invention described hereinabove and hereinbelow. The same is true for the iodine-modified cyclopeptides Tyr₂, FRGD and YRGD. That is, further building blocks of the invention are cyclo(3-I-YRGDLAYp(NMe)K); cyclo(3-I-YRGDLA3-I-Yp(NMe)K); cyclo(YRGDLA3-I-Yp(NMe)K); cyclo(3-I-YRGDLAFp(NMe)K); cyclo(FRGDLA3-I-Yp(NMe)K);

wherein 3-I-Y represents a Tyr residue that carries an iodine atom in the 3-position of the phenyl ring, wherein said iodine atom can be any non-radioactive isotope or radioisotope of iodine.

### Synthesis of Peptide

The cyclopeptides of the invention can be synthesized using standard peptide methodology such as solid phase peptide synthesis using Fmoc as a protective group. The available techniques are described for instance in J. Chatterjee, B. Laufer, H. Kessler, Nat. Protoc. 2012, 7, 432-444 and in WO 2017/046416 A.

Cyclization of the peptide can be effected using standard techniques. For instance, cyclization can be accomplished on the solid support or in solution using HBTU/HOBt/DIEA, PyBop/DIEA or PyClock/DIEA reagents. The available cyclization methods are described for instance in WO 2017/046416 A, J. Chatterjee, B. Laufer, H. Kessler, Nat. Protoc. 2012, 7, 432-444 and references cited therein.

### Synthesis of Conjugate

The conjugate may be prepared by analogous use of methods described in the literature.^{38,43,44,45}

### Diseases Associated with Cells Having Increased Expression of αvβ6-Integrin

The conjugates of the present invention are useful for any disease that is associated with an increased expression of αvβ6-integrin. Generally, the presence of αvβ6-integrin in tissue can be determined by immunohistochemistry (IHC). Applying this analytical technique to healthy adult tissue does not give rise to any αvβ6-integrin signal. Hence, in the context of some embodiments of the present invention, tissue giving rise to a detectable IHC signal for αvβ6-integrin is regarded as tissue with increase expression of αvβ6-integrin. Any tissue exhibiting increased expression of αvβ6-integrin is tissue deviating from healthy adult tissue, be it due to a disease such as cancer, fibrosis or Covid-19, or due to a condition like an earlier wound resulting in scar tissue formation. Any of these diseases and conditions may be identified using the conjugates of the present invention. Such diseases are described in the literature.^{41,42}

These diseases include cancer and especially non-small-cell lung cancer (NSCLC), pancreatic cancer, cholangiocellular cancer, gastric cancer, breast cancer, head-and-neck squamous cell, basal cell, colon cancer, ovarian cancer (Niu J, Li Z, Cancer Left. 2017; 403:128e137), and cancer of the upper aerodigestive tract and particularly pancreatic ductal adenocarcinoma (PDAC) (Sipos et al., Histopathol. 2004; 45:226, Reader C S, et al., J. Pathol. 2019; 249:332, Steiger K, et al., Mol. Imaging 2017; 16:1536012117709384). Of particular interest are lung adenocarcinoma, mammary carcinoma, colon adenocarcinoma, pancreatic adenocarcinoma (PDAC), head and neck squamous cell carcinoma such as oral squamous cell carcinoma, laryngeal squamous cell carcinoma, oropharyngeal squamous cell carcinoma, nasopharyngeal squamous cell carcinoma, hypopharyngeal squamous cell carcinoma.

Using IHC, αvβ6 expression in fibrotic tissue was also confirmed (Munger C S, et al., Cell 1999; 96:319). Further diseases therefore include fibrosis and especially biliary, renal, endomyocardial fibrosis, Crohn's disease, arthrofibrosis as well as pulmonary fibrosis. Of particular interest is idiopathic pulmonary fibrosis (IPF).

Quantification of αvβ6-integrin in lung tissue has been identified as a potentially valuable method for
(1) stratification of patients eligible for an inhalation therapy with an αvβ6-inhibitor molecule (e.g., GSK3008348) and
(2) evaluation of therapeutic success of such therapy (P. T. Lukey et al., European Journal of Nuclear Medicine and Molecular Imaging (2020) 47:967-979, https://doi.org/10.1007/s00259-019-04586-z; A. E. John et al., Nature Communications (2020)11:4659, https://doi.org/10.1038/s41467-020-18397-6 and T. M. Maher et al., Respiratory Research (2020) 21:75, https://doi.org/10.1186/s12931-020-01339-7). Hence, the present invention might be particularly suited for this and related fields of application.

A recent study suggested an expression of αvβ6 in lung tissue affected by COVID-19 (Foster C C, et al., J. Nucl. Med. 2020; 61:1717). The radiolabeled compounds of the present invention are therefore suitable for in-vivo imaging of post-COVID-19 syndrome in patients.

Since αvβ6-integrin is an activator of transforming growth factor beta (TGF-beta), any disease associated with abnormal TGF-beta levels in the intracellular space, or associated with a disturbed TGF-beta response of certain cell types resulting in altered TGF-beta signaling pathways, may be related to enhanced αvβ6-integrin expression. Such diseases could be diagnosed by determining the αvβ6-integrin expression status of cells in the affected tissues. Of particular interest is the use of diagnostic procedures based on the determination of αvβ6-integrin expression density in tissues for therapeutic decisions related to the use of therapeutic agents, above all, antibodies, targeting the TGF-beta signaling pathway, above all, TGF-beta itself in its free form or in form of its complex with latency-associated peptide.

Increased αvβ6 expression can be exploited for in-vivo targeting using radiolabeled compounds as those of the present invention.

### Use for Imaging and/or as Diagnostic Agent

Conjugates of the present invention are suitable for use as diagnostic agent. The conjugates of the present invention are advantageously used, wherein the effector moiety contains an active atom or active atomic group suitable for the imaging method/diagnostic method of interest, as described above. Depending on the chosen imaging/diagnostic method, a suitable active atom or active atomic group is selected. The chosen imaging/diagnostic method also determines the dosage, form and timing of the administration of the conjugate of the present invention.

The conjugates of the present invention are suitable for virtually any analytical/diagnostic method that involves the use of diagnostic agents. The conjugates of the present invention are particularly suitable for imaging methods such as gamma scintigraphy, fluorescence-based imaging, positron emission tomography (PET), single-photon emission computed tomography (SPECT), magnet resonance tomography (MRT), optical imaging or magnetic resonance imaging (MRI), X-ray based CT imaging, scintigraphy, Cherenkov imaging, ultrasonography, thermography and combinations thereof.

The conjugates of the present invention may be used by applying techniques described in the literature.^{33,34,35,38} The present invention thus provides methods for imaging patients, such as cancer patients, fibrosis patients or patients affected by Covid-19 infection, including post-COVID-19 syndrome, which comprise administration of the conjugate of the present invention to the patient, followed by subjecting the patient to an imaging method selected from gamma scintigraphy, fluorescence-based imaging, positron emission tomography (PET), single-photon emission computed tomography (SPECT), magnet resonance tomography (MRT), optical imaging or magnetic resonance imaging (MRI), X-ray based CT imaging, scintigraphy, Cherenkov imaging, ultrasonography, thermography and combinations thereof, wherein the active atom or active atomic group is suitable for the selected imaging method and wherein the selected imaging method detects a signal resulting from the active atom or active atomic group.

### Use as Therapeutic Agent

The conjugates of the present invention having an effector moiety with an active atom or active atomic group derived from a drug can be used in the treatment of the diseases associated with upregulation of αvβ6-integrin, e.g. as listed above.

The conjugates of the present invention may be administered to the patient for instance by intravenous, transmucosal, transdermal, intranasal administration. Suitable dosages may be in the range of 0.1 to 1000 mg/day, preferably 0.1 to 10 mg/day. The conjugates of the present invention may be administered once daily, twice a day, three times a day, etc. for any period of time, wherein multiple periods of time may be interrupted by one or more periods of time where the compounds of the present invention are not administered.

The conjugates of the present invention may also be used as a component in combination therapy. They may be combined with one or more other therapeutic agents effective in the treatment of cancer such as the therapeutic agents listed above and/or below. Such combination therapy may be carried out by simultaneously or sequentially administering the two or more therapeutic agents.

It is also possible to use conjugates of the present invention, particularly those incorporating radionuclides emitting alpha or beta radiation, such as for example, ⁴⁷Sc, ⁶⁷Cu, ¹⁷⁷Lu, ⁹⁰Y, ²¹³Bi, ²²⁵Ac, ¹⁶¹Tb, ¹⁴⁹Tb, or ¹³¹I, for targeted radiotherapy.

The conjugates of the present invention may also be used for diagnosis or treatment of fibrosis. The conjugates of the present invention may be used for such purposes by any suitable administration form including intravenous, intra-arterial, transmucosal, pulmonary, and intranasal administration. Dosages and administration schemes can be the same as specified above for the treatment of cancer. Combination therapy is also possible, wherein the one or more other therapeutic agents is selected from other therapeutic agents suitable for the treatment of fibrosis, for instance as cited above by cross-reference to the review article by Gharaee-Kermani et al. which is incorporated herein by reference. The conjugates of the present invention as well as the one or more other therapeutic agents can be administered simultaneously or sequentially.

The conjugates of the present invention may also be used for diagnosis or treatment of Covid-19 infections, including post-COVID-19 syndrome. The conjugates of the present invention may be used for such purposes by any suitable administration form including intravenous, intra-arterial, transmucosal, pulmonary, and intranasal administration. Dosages and administration schemes can be the same as specified above for the treatment of cancer. Combination therapy is also possible, wherein the one or more other therapeutic agents is selected from other therapeutic agents suitable for the treatment of Covid-19 infections, for instance immune therapy, dexamethasone or remdesivir. The conjugates of the present invention as well as the one or more other therapeutic agents can be administered simultaneously or sequentially.

The present invention thus provides methods for treating patients suffering from diseases associated with increased αvβ6 integrin expression, and especially cancer, fibrosis or Covid-19 infections, which include administration of a conjugate of the present invention to the patient, wherein the active atom or active atomic group is derived from a therapeutic agent that is selected to be suitable for treating the respective disease, e.g. as specified under Item (b-6) in the Effector Moiety section above.

### Use for Drug Targeting and in Biomolecular Research

The conjugates of the present invention may also be used for drug targeting as well as in biomolecular research. These uses may be carried out as described in the respective sections of WO 2017/046416 A. In particular, it is possible to covalently or non-covalently incorporate the conjugates of the present invention, preferably comprising a Tyr₂ peptide sequence, into nanocarriers such as nanoparticles, liposomes or micelles to allow the peptide moiety to bind to target cells to thereby increase local concentration of the nanoparticle, which typically contains a drug. This approach is of particular interest for the treatment of cancer and especially carcinoma with chemotherapeutics as it may accomplish a “homing” in such αvβ6-expressing tissues.

### Pharmaceutical Compositions

The conjugates of the present invention may be formulated as pharmaceutical compositions. This can be done using conventional means and methods for peptide-based medicaments. Suitable literature is recited for instance in the section on pharmaceutical compositions of WO 2017/046416 A. These disclosures are incorporated herein by reference. Pharmaceutical compositions of the present invention may also comprise the nanoparticles mentioned in the preceding section. According to a preferred embodiment, such nanoparticles comprise not only the conjugate of the present invention and the nanoparticle itself, but additionally also a therapeutic agent, preferably chemotherapeutic, within the nanoparticle.

### EXAMPLES

### Materials and Methods

### Abbreviations

CuAAC=copper-catalyzed azide-alkyne cycloaddition, Dde=1-(4,4-dimethyl-2,6-dioxocyclohex-1-ylidene)-3ethyl, DIAD=diisopropyl azodicarboxylate, DIPEA=N,N-diisopropylamine, DMF=dimethylformamide, DPPA=diphenyl phosphoryl azide, Fmoc=9-fluorenylmethoxycarbonyl, HATU=N,N,N′,N′,-tetramethyluronium-hexafluorophosphate, HFIP=1,1,1,3,3,3-hexafluoro-2-propanol, HOBt=1-hydroxybenzotriazole hydrate, NMP=N-methyl-2-pyrrolidone, NOTA=1,4,7-triazacyclononane-1,4,7-triacetic acid, Pbf=2,2,4,6,7-pentamethyldihydrobenzofuran-5-sulfonyl, PBS=phosphate-buffered saline, PPh₃=triphenylphosphine tBu=tert-Butyl, TFA=trifluoroacetic acid, THF=tetrahydrofuran, TIPS=triisopropyl silane, TRAP=1,4,7-triazacyclononane-1,4,7-tris[methylene(2-carboxyethylphosphinic acid)]

### General

Unless otherwise noted, all commercially available reagents and solvents were of analytical grade and were used without further purification. Protected amino acids were purchased from IRIS Biotech (Germany). Cu(OAc)₂·H₂O, 4-pentynoic acid, diisopropylamine (DIPEA) and sodium ascorbate were purchased from Sigma Aldrich (Darmstadt, Germany). 1,4,7-triazacyclononane-1,4,7-triacetic acid (NOTA) was purchased from Chematech (Dijon, France). HATU was obtained from Bachem Holding AG (Bubendorf, Switzerland). HOBt hydrate was obtained from Carbolution (St. Ingbert, Germany). TRAP(azide)₁³⁸ and TRAP(azide)₃⁴³ were synthesized as described previously. Semi-preparative reversed-phase HPLC was performed using a Waters system: Waters 2545 (Binary Gradient Module), Waters SFO (System Fluidics Organizer), Waters 2996 (Photodiode Array Detector) and Waters 2767 (Sample Manager). Separations were performed using a Dr. Maisch C18-column: Reprosil 100 C18, 5 μm, 150×30 mm (Column 1) with a flow rate of 40 mL/min of water (0.1% v/v trifluoroacetic acid and acetonitrile (0.1% v/v trifluoroacetic acid) or a YMC C18-column: YMC-Pack ODS-A, 5 μm, 250×20 mm (Column 2) with a flow rate of 16 mL/min of water (0.1% v/v trifluoroacetic acid) and acetonitrile (0.1% v/v trifluoroacetic acid). Analytical HESI-HPLC-MS (heated electrospray ionization mass spectrometry) was performed on a LCQ Fleet (Thermo Scientific) with a connected UltiMate 3000 UHPLC focused (Dionex) on C18-columns: 51: Hypersil Gold aQ 175 Å, 3 μm, 150×2.1 mm (for 8 or 20 minutes measurements); S2: Accucore C18, 80 Å, 2.6 μm, 50×2.1 mm (for 5 minute measurements) (Thermo Scientific). Linear gradients (5%-95% acetonitrile content) with water (0.1% v/v formic acid) and acetonitrile (0.1% v/v formic acid) were used as eluents. The affinity and selectivity of integrin ligands were determined by a solid-phase binding assay, applying a previously described protocol,⁴⁴ whereby compounds containing a metal binding unit (a chelator, e.g., TRAP) were previously transformed into the Ga^{III} complexes by addition of an equimolar amount of aq Ga(NO₃)₃.

### Example 1: Peptide Synthesis Procedure

Carried out according to a previously established protocol with the exception of performing the synthesis in DMF in place of N-methyl-2-pyrrolidone (NMP).⁴⁴

Loading of the CTC-resin. Peptide synthesis was carried out using a CTC-resin (0.9 mmol/g) following a standard Fmoc-protected peptide strategy. Fmoc-Xaa-OH (1.5 eq.) were attached to the CTC-resin with N,N-diisopropylamine (DIPEA, 2.5 eq.) in anhydrous DCM (0.8 mL/g (resin)) at rt for 1 h. Capping of the remaining trityl-chloride groups was performed by addition of a solution of MeOH (1 mL/g (resin)) and DIPEA (5:1, v/v) for 15 min. The resin was filtered and washed with DCM (5×) and with MeOH (3×).

On-Resin Fmoc-Deprotection. The Fmoc-protected peptidyl-resin was treated with a 20% piperidine in DMF (v/v) for 10 min and again for 5 min. The resin was washed with DMF (5×).

Standard Amino Acid Coupling. A solution of Fmoc-Xaa-OH (2 eq.), HATU (2 eq.), HOBt (2 eq.) and DIEA (3 eq.) in DMF (1 mL/g (resin)) was added to the free amino peptidyl-resin and shaken for 1 h at rt. Solution was washed with DMF (5×). Complete coupling was monitored by analytical RP-HPLC and MS. A small amount of resin was dissolved in a solution of 20% HFIP in DCM followed by a small amount of MeOH and MeCN. Solution was filtered and analysed by RP-HPLC and MS.

On-Resin N-Methylation. The linear Fmoc-deprotected peptide was treated with a solution of 2-nitrobenzenesulfonylchloride (o-Ns-Cl, 4 eq.) and 2,4,6-Collidine (10 eq.) for 20 min at rt. Resin was washed with DCM (3×) and THF (5×). A solution of triphenylphosphine (PPh₃, 5 eq.) in anhydrous MeOH and a solution of diisopropyl azodicarboxylate (DIAD, 5 eq.) in a minimum amount of THF was prepared and added to the resin. Resin solution was shaken for 15 min before washing with THF (5×) and DMF (5×).

Cleavage of Linear Peptides from Resin. Peptidyl-resin was treated with a solution of 20% HFIP in DCM (3×30 min) to ensure complete cleavage of the peptide from the resin before solvent evaporation under pressure.

Cyclization of Linear Peptide. Peptide was dissolved in DMF (1 mM peptide concentration) before addition of NaHCO3 (5 eq.) and DPPA (3 eq.). Reaction occurred at rt with stirring overnight where cyclization was monitored by RP-HPLC and MS. Solvent was evaporated to a small volume under pressure, filtered through glass wool and solvent evaporation continued.

Cleavage of Dde-Protection Group. The cyclized peptide was dissolved in DMF before addition of Hydrazine Hydrate (2% v/v). Reaction occurred with stirring for 30 min at rt. Dde-deprotection was monitored by HPLC-MS

Cleavage of acid-labile protection groups. The cyclized peptide was dissolved in a 10:85:2.5:2.5 (DMF:TFA:TIPS:H₂O) solution for 1 hr. De-protection was monitored by HPLC-MS.

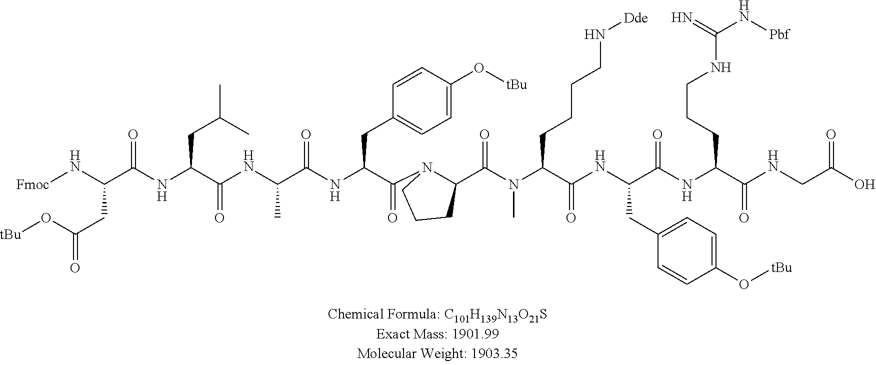

Structural Formula of the Linear Peptide Y(tBu)R(tBu,Fmoc)GD(Pbf)LAY(tBu)p(NMe)K(Dde).

Synthesis of Y(tBu)R(tBu,Fmoc)GD(Pbf)LAY(tBu)p(NMe)K(Dde). The linear protected peptide Y(tBu)R(tBu,Fmoc)GD(Pbf)LAY(tBu)p(NMe)K(Dde) was synthesised according to the above procedure. Formation of the complete linear sequence was monitored by HPLC-MS (m/z: 1903.00 [M+H⁺]⁺, 952.08 [M+2H⁺]²⁺).

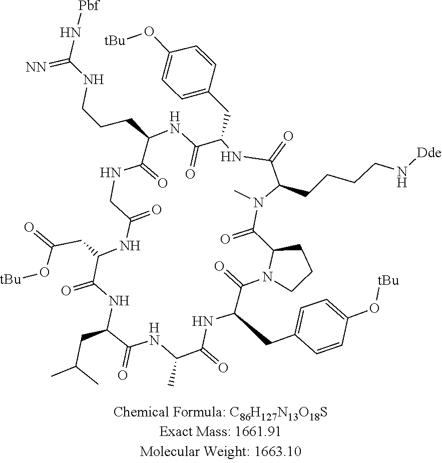

Structural Formula of the Protected Cyclic Peptide Cyclo(Y(tBu)R(Pbf)GD(tBu)LAY(tBu)p(NMe)K(Dde)).

Synthesis of cyclo(Y(tBu)R(PbOGD(tBu)LAY(tBu)p(NMe)K(Dde)). The cyclic protected peptide cyclo(Y(tBu)R(Pbf)GD(tBu)LAY(tBu)p(NMe)K(Dde)) was synthesised according to the above procedure. The cyclisation was performed without any prior HPLC purification of the linear peptide. Formation of the cyclised peptide was monitored by HPLC-MS (m/z: 1663.17 [M+H⁺]⁺, 832.08 [M+2H⁺]²⁺).

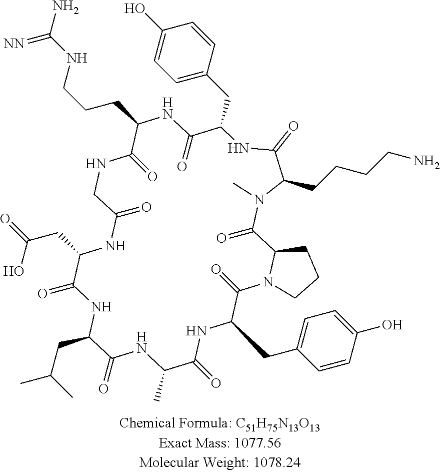

Structural Formula of Tyr₂ [Cyclo(YRGDLAYp(NMe)K)].

Synthesis of Tyr₂. Cleavage of the Dde protecting group from cyclo(Y(tBu)R(Pbf)GD(tBu)LAY(tBu)p(NMe)K(Dde)) was performed as described above. Cyclo(Y(tBu)R(Pbf)GD(tBu)LAY(tBu)p(NMe)K) was obtained as a white solid with a yield of 35% (508.7 mg, 339.4 μmol) (relating to the loading capacity of the resin). RP-HPLC (gradient: 20-60% MeCN in H₂O containing 0.1% TFA, in 25 min): t_{R}=10.35 min (column 1). Directly after Dde-deprotection, 78 mg of the crude material was dissolved in toluene (50 mL) toluene and rotary evaporated to remove any reagents from the Dde-deprotection. This resulted in a orange/brown oil that was directly treated with a 2 ml acid-labile deprotecting solution described above. The cyclic peptide Tyrₑ [cyclo(YRGDLAYp(NMe)K)] was obtained as a colorless solid with a yield of 10.2% (In relation to the crude product) (5.75 mg, 5.33 μmol). RP-HPLC (gradient: 20-70% MeCN in H₂O containing 0.1% TFA, in 25 min): t_{R}=10.07 min (column 1). m/z: 540.14 [M+2H⁺]²⁺.

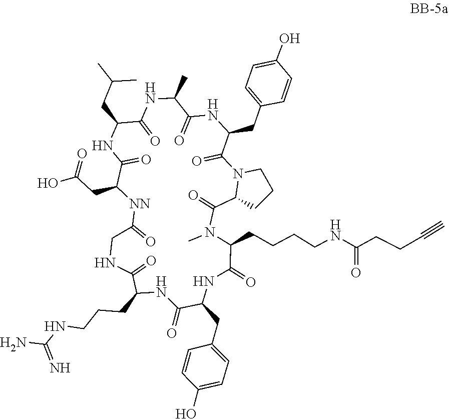

Synthesis of BB-5a. 4-Pentynoic acid (2.38 mg, 24.23 μmol, 1.2 eq), HATU (9.21 mg, 24.23 μmol, 1.2 eq), HOBt (3.3 mg, 24.23 μmol, 1.2 eq) and DIPEA (10.29 μL, 60.59 μmol, 3 eq) were dissolved in a minimum amount of DMF and allowed to react for 15 min before the dropwise addition to a solution of the dissolved Dde-deprotected peptide with acid-labile protecting groups (30.27 mg, 20.19 μmol, 1 eq) in DMF. The reaction occurred with stirring for 1 h. Conjugation of the alkyne functional group was monitored by HPLC-MS. The solvent was evaporated under pressure resulting in an orange/brown oil that was directly treated with the 2 mL acid-labile deprotecting solution described above. Cyclo(YRGDLAYp(NMe)K(pentynoic acid)), BB-5a, was obtained as colorless solid with a yield of 57% (13.26 mg, 11.45 μmol). RP-HPLC (gradient: 30-50% MeCN in H₂O containing 0.1% TFA, in 15 min): t_{R}=7.67 min (column 1). m/z: 1737.30 [3M+2H⁺]²⁺, 1158.51 [M+H⁺]⁺, 580.05 [M+2H⁺]²⁺

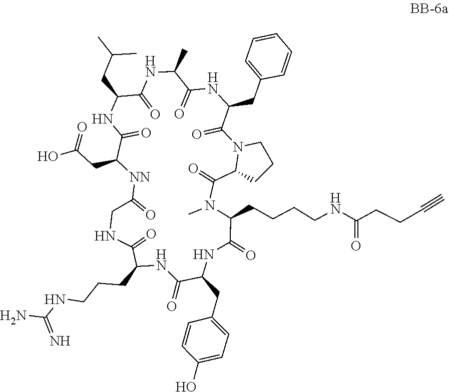

Synthesis of BB-6a. 4-Pentynoic acid (7.63 mg 77.79 μmol L5 eq), HATU (2166 mg, 62.23 μmol 1.2 eq), HOBt (9.53 mg, 62.23 μmol 12 eq) and DIPEA (27.1 μL, 155.58 μmol 3 eq) were dissolved in a minimum amount of DMF and allowing to react for 15 mins before the dropwise addition to a solution of the dissolved Dde-deprotected YRGD peptide with acid-labile protecting groups (73.99 mg, 51.86 μmol, 1 eq) in DMF. The solvent was evaporated under pressure resulting in an orange/brown oil that was directly treated with the 3 ml acid-labile deprotecting solution previously described. C-9 was obtained as a colourless solid with a yield of 76% (45 mg, 39.39 μmol). RP-HPLC (gradient: 30-80% MeCN in H₂O containing 0.1% TFA, in 20 min): t_{R}=9.4 min (column 1). m/z: 1164.41 [M+Na⁺+H⁺]⁺, 1142.46 [M+H]⁺, 572.11 [M+2H⁺]²⁺.

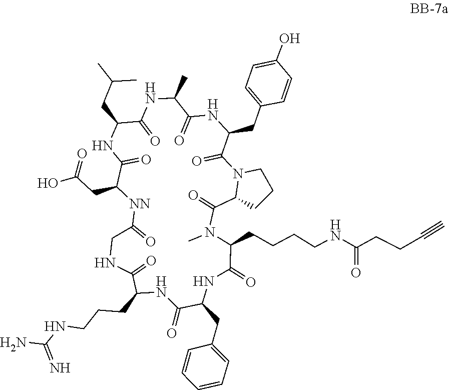

Synthesis of BB-7a. 4-Pentynoic acid (3.05 mg 31.12 μmol 1.5 eq), HATU (9.47 mg, 24.9 μmol 1.2 eq), HOBt (3.81 mg, 24.9 μmol 1.2 eq) and DIPEA (10.84 μL, 62.24 μmol 3 eq) were dissolved in a minimum amount of DMF and allowing to react for 15 mins before the dropwise addition to a solution of the dissolved Dde-deprotected FRGD peptide with acid-labile protecting groups (29.6 mg, 20.75 μmol, 1 eq) in DMF. The solvent was evaporated under pressure resulting in an orange/brown oil that was directly treated with the 2 ml acid-labile deprotecting solution previously described. C-8 was obtained as a colourless solid with a yield of 28.2% (6.68 mg, 5.85 μmol). RP-HPLC (gradient: 30-80% MeCN in H₂O containing 0.1% TFA, in 20 min): t_{R}=8.9 min (column 1). m/z: 1165.09 [M+Na⁺+H⁺]⁺, 1142.47 [M+H⁺]⁺, 572.21 [M+2H⁺]²⁺.

Synthesis of C-1. Cyclo(YRGDLAYp(NMe)K(pentynoic acid)) (8.01 mg, 6.92 μmol, 1.5 eq) was added to a solution a TRAP(azide)₁ (3.05 mg, 4.61 μmol, 1 eq) and sodium ascorbate (45.7 mg, 230.5 μmol, 50 eq) in a minimum amount of H₂O. Copper(II) acetate (1.1 mg, 5.53 μmol, 1.2 eq) was added and a brown precipitate immediately formed. Upon vortexing, the solution turned to a transparent green. The solution reacted for 1 h at 60° C. without stirring. After 1 h, Cu demetallation of the peptidyl-chelator compound was done by addition of 1,4,7-triazacyclononane-1,4,7-triacetic acid (NOTA) (41.94 mg, 138.26 μmol, 30 eq.) dissolved in water (1 mL) with adjustment of pH to 2.2 by addition of 1 M aq HCl. The mixture was reacted for 1 h at 60° C. Synthesis of TRAP(Tyr₂) was monitored by HPLC-MS. C-1 was obtained as a colorless solid with a yield of 5.7% (0.48 mg, 0.26 μmol). RP-HPLC (gradient: 20-70% MeCN in H₂O containing 0.1% TFA, in 25 min): t_{R}=12.3 min (column 1). m/z: 910.49 [M+2H⁺]²⁺, 607.73 [M+3H⁺]³⁺.

Synthesis of C-7. Cyclo(YRGDLAYp(NMe)K(pentynoic acid)) (24.96 mg, 21.55 μmol, 3.3 eq) was added to a solution of TRAP(azide)₃ (5.39 mg, 6.53 μmol, 1 eq) and sodium ascorbate (64.7 mg, 326.6 μmol, 50 eq) in a minimum amount of H₂O. Copper(II) acetate (1.56 mg, 7.84 μmol, 1.2 eq) was added and a brown precipitate immediately formed. Upon vortexing, the solution turned to a transparent green. The solution reacted for 1 h at 60° C. without stirring. After 1 h, Cu demetallation of the peptidyl-chelator compound was done by addition of 1,4,7-triazacyclononane-1,4,7-triacetic acid (NOTA) (39.6 mg, 130.6 μmol, 20 eq.) dissolved in water (1 mL) with adjustment of pH to 2.2 by addition of 1 M aq HCl. Mixture was reacted for 1 h at 60° C. Synthesis of TRAP(Tyr₂)₃ was monitored by HPLC-MS. C-7 was obtained as colorless solid with a yield of 36.1% (10.11 mg, 2.35 μmol). RP-HPLC (gradient: 20-40% MeCN in H₂O containing 0.1% TFA, in 15 min followed by a 6 min washing phase (100% MeCN): t_{R}=17.35 min (column 2). m/z: 1434.01 [M+3H⁺]³⁺, 1075.97 [M+4H⁺]⁴⁺, 861.03 [M+5H⁺]⁵⁺.

Synthesis of C-8. BB-7a (6 mg, 5.25 μmol, 3.3 eq) was added to a solution of TRAP(azide)₃ (1.3 mg, 1.6 μmol, 1 eq) and sodium ascorbate (15.8 mg, 79.6 μmol, 50 eq) in a minimum amount of H₂O:tBuOH, 4:1. Copper(II) acetate (381.3 μg, 1.91 μmol, 1.2 eq) was added and a brown precipitate immediately formed. Upon vortexing, the solution turned to a transparent green. The solution reacted for 1 h at 60° C. without stirring. After 1 h, formation of C-8 was monitored by HPLC-MS. Cu removal of the peptidyl-chelator compound was performed by addition of 1,4,7-triazacyclononane-1,4,7-triacetic acid (NOTA) (14.5 mg, 47.8 μmol, 30 eq.) dissolved in water (0.5 mL) with adjustment of pH=2.2. Mixture was reacted for 1 h at 60° C. C-8 was obtained as a colourless solid with a yield of 42.9% (2.9 mg, 0.7 μmol). RP-HPLC (gradient: 10-70% MeCN in H₂O containing 0.1% TFA, in 20 min): t_{R}=19.2 min (column 1). m/z: 1426.38 [M+Na⁺+3H⁺]³⁺, 1070.15 [M+Na⁺+4H⁺]⁴⁺, 856.34 [M+Na⁺+5H⁺]⁵⁺, 713.74 [M+Na⁺+6H⁺]⁶⁺.

Synthesis of C-9. BB-6a (45 mg, 39.39 μmol, 3.3 eq) was added to a solution of TRAP(azide)₃ (9.86 mg, 11.94 μmol, 1 eq) and sodium ascorbate (118.24 mg, 596.9 μmol, 50 eq) in a minimum amount of H₂O:tBuOH, 4:1. Copper(II) acetate (2.86 mg, 14.32 μmol, 1.2 eq) was added and a brown precipitate immediately formed. Upon vortexing, the solution turned to a transparent green. The solution reacted for 1 h at 60° C. without stirring. After 1 h, formation of C-9 was monitored by HPLC-MS. Cu removal of the peptidyl-chelator compound was performed by addition of 1,4,7-triazacyclononane-1,4,7-triacetic acid (NOTA) (110.7 mg, 365 μmol, 30 eq.) dissolved in water (1 mL) with adjustment of pH=2.2. Mixture was reacted for 1 h at 60° C. C-9 was obtained as a colourless solid with a yield of 24.7% (12.78 mg, 3.01 μmol). RP-HPLC (gradient: 10-70% MeCN in H₂O containing 0.1% TFA, in 20 min): t_{R}=19.5 min (column 1). m/z: 1426.11 [M+Na⁺+3H⁺]³⁺, 1070.11 [M+Na⁺+4H⁺]⁴⁺, 856.38 [M+Na⁺+5H⁺]⁵⁺, 713.68 [M+Na⁺+6H⁺]⁶⁺

Synthesis of C-10 and C-11. The building block AvB6 (as described in Maltsev et al.³⁸) (6.08 mg, 5.4 μmol, 1 eq) was added to a solution a TRAP(azide)₃ (4.46 mg, 5.4 μmol, 1 eq) and sodium ascorbate (53.47 mg, 269.90 μmol, 50 eq) in a minimum amount of H₂O. Copper(II) acetate (1.29 mg, 6.48 μmol, 1.2 eq) was added and a brown precipitate immediately formed. Upon vortexing, the solution turned to a transparent green. The solution reacted for 1 h at 60° C. without stirring. BB-5a (13.75 mg, 11.87 μmol, 2.2 eq) was added directly into the reaction mixture and reacted for a further 1 h at 60° C. without stirring. After 1 h, Cu demetallation of the peptidyl-chelator compound was performed by addition of 1,4,7-triazacyclononane-1,4,7-triacetic acid (NOTA) (48.62 mg, 160.31 μmol, 30 eq.) dissolved in water (1 mL) with adjustment of pH=2.2. Mixture was reacted for 1 h at 60° C. Formation of C-11 and C-10 were monitored by HPLC-MS.

C-10 was obtained as colorless solid with a yield of 6.8% (1.55 mg, 0.37 μmol). RP-HPLC (gradient: 40-95% MeCN in H₂O containing 0.1% TFA, in 30 min): t_{R}=10.6 min (column 1). m/z: 1424.0 [M+3H⁺]³⁺, 1067.9 [M+2H⁺]⁴⁺, 854.8 [M+4H⁺]⁵⁺.

C-11 was obtained as colorless solid with a yield of 8.75% (2 mg, 0.47 μmol). RP-HPLC (gradient: 40-95% MeCN in H₂O containing 0.1% TFA, in 30 min): t_{R}=14.9 min (column 1). m/z: 1413.2 [M+3H⁺]³⁺, 1059.9 [M+2H⁺]⁴⁺, 848.2 [M+4H⁺]⁵⁺.

### Radiochemistry

Radiometal incorporation and radiochemical purity of labeled compounds was determined by radio-TL on ITLC silica impregnated chromatography paper (Agilent, Santa Clara, USA; eluents: 0.1 M trisodium citrate or a 1:1 (v/v) mixture of 1 M ammonium acetate and methanol), analyzed using a scan-RAM radio-TLC detector by LabLogic systems Inc. (Brandon, USA). ⁶⁸Ga-labelling was performed using a fully-automated on-site system (GallElut⁺ by Scintomics, Lindach, Germany) as described previously.⁴⁵ Briefly, the eluate of a ⁶⁸Ge/⁶⁸Ga-generator with SnO₂ matrix (by IThemba LABS, SA; 1.25 mL, eluent: 1 M aq. HCl, containing approx. 500 MBq ⁶⁸Ga) was adjusted to pH 2 by addition of aq. HEPES buffer (450 μL, 2.7 M) and applied for labeling of 5 nmol of a chelator conjugate for 2 min at 95° C. The radiolabeled peptides were trapped on Sep-Pak® C8 light solid phase extraction (SPE) cartridges, which were purged with water (10 mL). The product was eluted with 2 mL aq. EtOH (50%). After evaporation of the ethanol, the purity was determined by radio-TLC and was always found to be ≥98%.

### Example 2: Activity Assessment

### Determination of Log D Value

For the determination of n-octanol-PBS distribution coefficients (log D_{7.4}), 500 μL 1-octanol and 500 μL phosphate buffered saline were combined in a 1.5 mL Eppendorf tube. Approx. 1 MBq of the radiolabeled compound was added and vortexed vigorously for three minutes. The samples were centrifuged (13.000 rpm, 5 min) and the activities in 200 μL of the organic phase and 20 μL of the aqueous phase were quantified in a γ-counter.

### Cell Lines and Animal Models

All animal studies have been performed in accordance with general animal welfare regulations in Germany and the institutional guidelines for the care and use of animals. H2009 human lung adenocarcinoma cells (CRL-5911; American Type Culture Collection) were cultivated as recommended by the distributor. To generate tumor xenografts, 6- to 8-week-old female CB17 SCID mice (Charles River) were inoculated with 10⁷ H2009 cells in Matrigel (CultrexBME, type 3 PathClear; Trevigen, GENTAUR GmbH). Mice were used for biodistribution or PET studies when tumors had grown to a diameter of 10-12 mm (4-6 weeks after inoculation).

### PET Imaging

Mice were anaesthetized with isoflurane for intravenous administration of the radiolabeled compounds. The administered activity per mouse ranged between 10 and 15 MBq (100-200 pmol, depending on variations in timing of production and administration). PET imaging was performed on a Siemens Inveon small-animal PET system, either dynamic under isoflurane anaesthesia for 90 min, or as single frames 75 min p.i. with an acquisition time of 20 min. Data were reconstructed using Siemens Inveon Research Workspace software, employing a three-dimensional ordered subset expectation maximum (OSEM3D) algorithm without scatter and attenuation correction. For kinetic analyses, regions of interest (ROIs) were defined manually.

### Biodistribution

For biodistribution studies, 3-6 MBq (between 70-140 pmol) of the radiolabeled compound was injected into the tail vein. The mice were sacrificed 90 min after injection, a blood sample was taken and the organs of interest were dissected. Quantification of the activity in weighed tissue samples was done using a 2480 WIZARD² automatic γ-counter (PerkinElmer, Waltham, USA). Injected dose per gram tissue (% ID/g) was calculated from the organ weights and counted activities.

### Results

Novel peptidic compounds and conjugates were synthesized and characterized as described above.

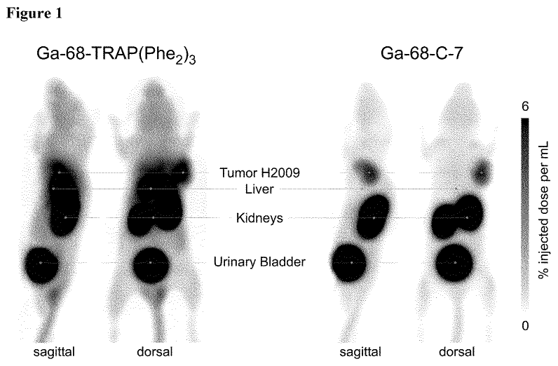

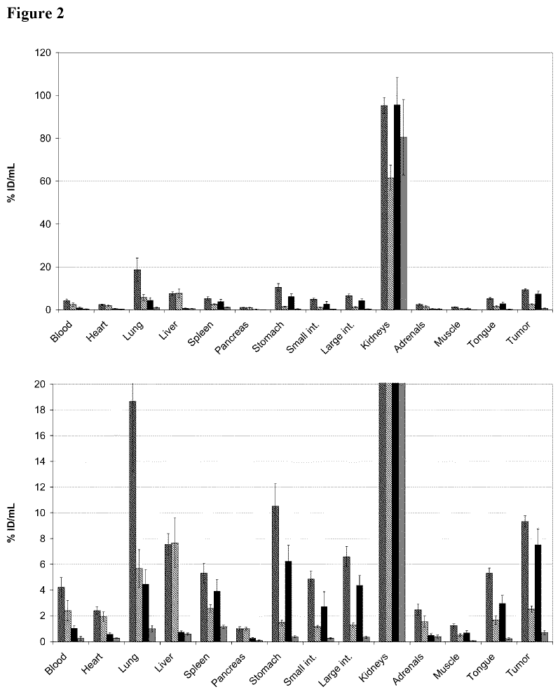

⁶⁸Ga-labeled trimeric conjugates of Phe₂ and Tyr₂, Ga-68-TRAP(Phe₂)₃³⁸ and Ga-68-C-7, were evaluated in H2009 tumor bearing mice. A comparison of the PET images (FIG. 1) shows that a low background activity and a clear delineation of the tumor is achieved with Ga-68-C-7, but not with Ga-68-TRAP(Phe₂)₃, mainly caused by a strong uptake in the liver. The corresponding ex-vivo biodistribution data (FIG. 2) confirm a high level of accumulation of Ga-68-TRAP(Phe₂)₃ in the liver. Since this uptake is not reduced by co-injection of a high excess (50 nmol) of unlabeled TRAP(Phe₂)₃ (blockade), it is proven not to be target-specific. Surprisingly, replacement of Phe by Tyr in Ga-68-C-7 reduced this unspecific uptake to insignificance, and also reduced unspecific uptakes in other compartments and tissues, namely, blood, heart, spleen, and tumor, ultimately resulting in superior PET image contrast as depicted in FIG. 1

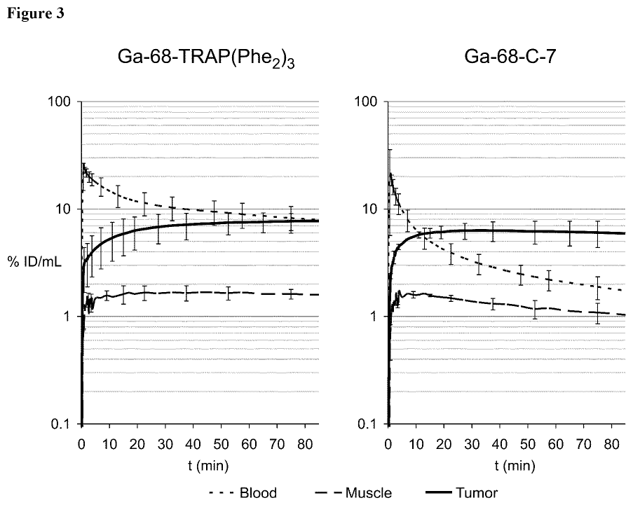

While the analysis of the biokinetics (FIG. 3) indicates a good tumor retention of both compounds, Ga-68-C-7 is cleared much more rapidly from the blood pool, ultimately resulting in a lower background in the PET images as depicted in FIG. 1

In summary, Ga-68-C-7 shows markedly improved biokinetics and imaging properties in comparison to the corresponding state-of-the-art compound, Ga-68-TRAP(Phe₂)₃,³⁸ substantiating that Tyr₂ is advantageously used in αvβ6-integrin targeted compounds for in-vivo applications.

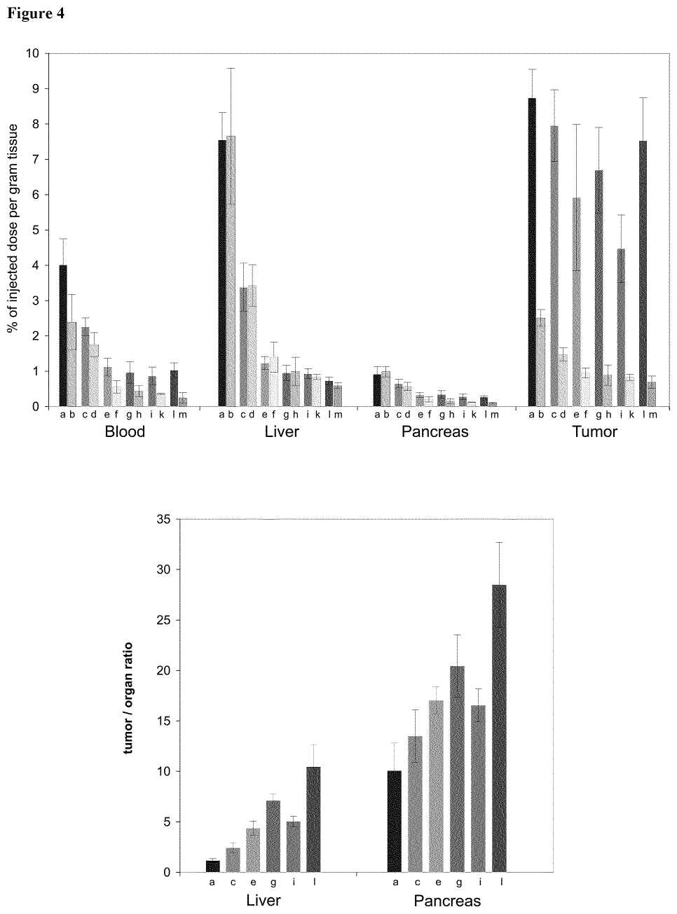

The biodistribution of ⁶⁸Ga-labeled trimeric TRAP conjugates comprising different combinations of Phe₂, FRGD, YRGD, and Tyr₂, namely, Ga-68-TRAP(Phe₂)₃, Ga-68-C-7, Ga-68-C-8, Ga-68-C-9, Ga-68-C-10, and Ga-68-C-11, were evaluated in H2009 tumor bearing mice. FIG. 4 shows that even exchanging one single Phe₂ in the structure of Ga-68-TRAP(Phe₂)₃ by a Tyr₂, resulting in Ga-68-C-11, markedly reduces unspecific liver uptake (non-specificity proven by similarity of control vs. blockade experiments), reduces remaining activity in blood, reduces pancreatic uptake, while Ga C-10 still shows a high tumor uptake. Exchange of two Phe₂ in the structure of Ga-68-TRAP(Phe₂)₃ by Tyrₑ, resulting in Ga-68-C-10, has a similar effect, albeit even more pronounced. Likewise, exchange of all Phe₂ in the structure of Ga-68-TRAP(Phe₂)₃ by FRGD or YRGD, resulting in Ga-68-C-8 and Ga-68-C-9, respectively, shows that the cyclopeptides comprising only one tyrosine show also superior properties. Of all investigated trimeric conjugates, Ga-68-C-7 shows the best tumor-to-liver and particularly tumor-to-pancreas ratios, suggesting that it should be most suitable for imaging αvβ6-integrin positive lesions in those organs, such as metastases or primary tumors of the pancreatic adenocarcinoma type.

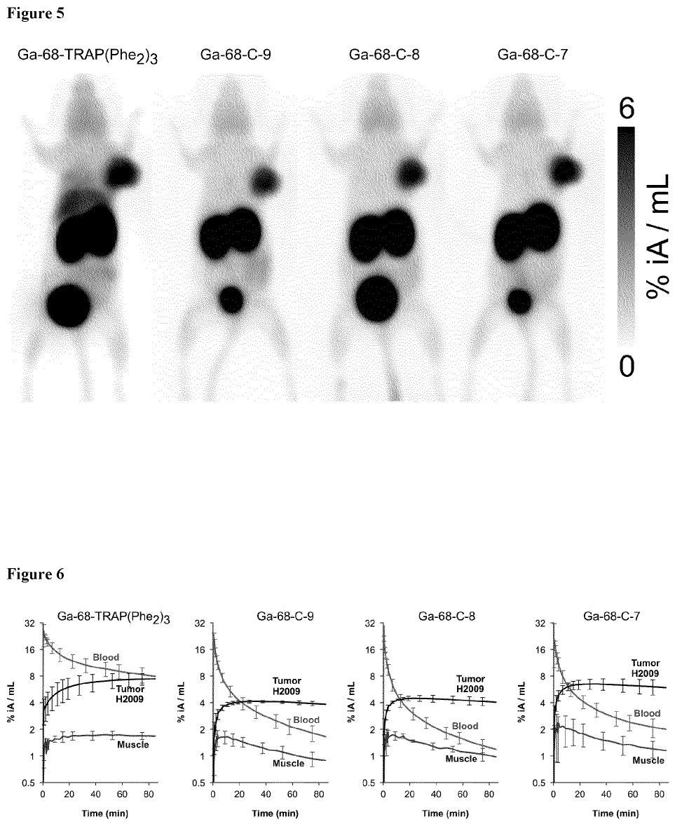

FIG. 5 corroborates that the peptides FRGD and YRGD, which are featured in Ga-68-C-8 and Ga-68-C-9, respectively, are also suitable for synthesis of targeted radiolabeled molecules with significantly lower liver uptake than Ga-68-TRAP(Phe₂)₃. Accordingly, FIG. 6 shows that the blood clearance of Ga-68-C-8 and Ga-68-C-9 is much faster than that of Ga-68-TRAP(Phe₂)₃, and resembles that of Ga-68-C-10.

### REFERENCES

1 Margadant C, Monsuur H N, Norman J C, Sonnenberg A. Mechanisms of integrin activation and trafficking. Curr Opin Cell Biol. 2011:23; 607-614.
2 Avraamides C J, Garmy-Susini B, Varner J A. Integrins in angiogenesis and lymphangiogenesis. Nat Rev Cancer. 2008:8; 604-617.
3 Breuss J M, Gillett N, Lu L, Sheppard D, Pytela R. Restricted distribution of integrin β6 mRNA in primate epithelial tissues. J Histochem Cytochem. 1993:41; 1521-1527.
4 Niu G, Chen X. Why integrin as a primary target for imaging and therapy. Theranostics. 2011:1; 30-45.
5 Bandyopadhyay A, Raghavan S. Defining the role of integrin αvβ6 in cancer. Curr Drug Targets. 2009:10; 645-652.
6 Sipos B, Hahn D, Carceller A, et al Immunohistochemical screening for β6-integrin subunit expression in adenocarcinomas using a novel monoclonal antibody reveals strong up-regulation in pancreatic ductal adenocarcinomas in vivo and in vitro. Histopathol. 2004; 45:226-236.
7 Patsenker E, Wilkens L, Banz V, et al. The αvβ6 integrin is a highly specific immunohistochemical marker for cholangiocarcinoma. J Hepatol. 2010; 52:362-369.
8 Kawashima A, Tsugawa S, Boku A, et al. Expression of alphav integrin family in gastric carcinomas: increased αvβ6 is associated with lymph node metastasis. Pathol Res Pract. 2003; 199:57-64.
9 Zhang Z Y, Xu K S, Wang J S, et al. Integrin αvβ6 acts as a prognostic indicator in gastric carcinoma. Clin Oncol. 2008; 20:61-66.
10 Arihiro K, Kaneko M, Fujii S, Inai K, Yokosaki Y. Significance of alpha 9 beta 1 and alpha v beta 6 integrin expression in breast carcinoma. Breast Cancer. 2000; 7:19-26.
11 Ahmed N, Pansino F, Clyde R, et al. Overexpression of αvβ6 integrin in serous epithelial ovarian cancer regulates extracellular matrix degradation via the plasminogen activation cascade. Carcinogenesis. 2002; 23:237-244.
12 Ahmed N, Riley C, Rice G E, Quinn M A, Baker S. αvβ6 integrin—a marker for the malignant potential of epithelial ovarian cancer. J Histochem Cytochem. 2002; 50:1371-1380.
13 Bates R C, Bellovin D I, Brown C, et al. Transcriptional activation of integrin β6 during the epithelial-mesenchymal transition defines a novel prognostic indicator of aggressive colon carcinoma. J Clin Invest. 2005; 115:339-347.
14 Ramos D M, But M, Regezi B L, et al. Expression of integrin β6 enhances invasive behavior in oral squamous cell carcinoma. Matrix Biol. 2002; 21:297-307.
15 Steiger K, et al., Molecular Imaging, 2017, 16:1-3, DOI: 10.1177/1536012117709384
16 Wang B, Dolinski B M, Kikuchi N, et al. Role of αvβ6 integrin in acute biliary fibrosis. Hepatology. 2007; 46:1404-1412.
17 Hahm K, Lukashev M E, Luo Y, et al. αvβ6 integrin regulates renal fibrosis and inflammation in Alport mouse. Am J Pathol. 2007; 170:110-125.
18 Horan G S, Wood S, Ona V, et al. Partial inhibition of integrin αvβ6 prevents pulmonary fibrosis without exacerbating inflammation. Am J Respir Crit Care Med. 2008; 177:56-65.
19 Goodman S L, Hölzemann G, Sulyok G A, Kessler H. Nanomolar small molecule inhibitors for αvβ6, αvβ5, and αvβ3 integrins. J Med Chem. 2002; 45:1045-1051.
20 Kraft S, Diefenbach B, Mehta R, Jonczyk A, Luckenbach G A, Goodman S L. Definition of an unexpected ligand recognition motif for αvβ6 integrin. J Biol Chem. 1999; 274:1979-1985.
21 Hausner S L, DiCara D. Marik J, Marshall J F, Sutcliffe J F. Use of a peptide derived from foot-and-mouth disease virus for the noninvasive imaging of human cancer: generation and evaluation of 4-[¹⁸F]fluorobenzoyl A20FMDV2 for in vivo imaging of integrin αvβ6 expression with positron emission tomography. Cancer Res. 2007; 67:7833-7840.
22 Li, S, Mcguire, K T, Lin M, et al. Synthesis and characterization of a high-affinity αvβ6-specific ligand for in vitro and in vivo applications. Mol Cancer Ther. 2009; 8:1239-1249.
23 Kimura R H, Teed R, Hackel B J, et al. Pharmacokinetically stabilized cystine knot peptides that bind alpha-v-beta-6 integrin with single-digit nanomolar affinities for detection of pancreatic cancer. Clin Cancer Res. 2012; 18:839-849.
24 Liu H, Wu Y, Wang F, Liu Z. Molecular imaging of integrin αvβ6 expression in living subjects. Am J Nucl Med Mol Imaging. 2014; 4:333-345.
25 John A E, Luckett J C, Tatler A L, et al. Preclinical SPECT/CT imaging of αvβ6 integrins for molecular stratification of idiopathic pulmonary fibrosis. J Nucl Med. 2013; 54:2146-2152.
26 Liu Z, Liu H, Ma T, et al. Integrin αvβ₆-Targeted SPECT Imaging for Pancreatic Cancer Detection. J Nucl Med. 2014; 55:989-994.
27 Zhu X, Li J, Hong Y, et al. ^{99m}Tc-labeled cystine knot peptide targeting integrin αvβ6 for tumor SPECT imaging. Mol Pharm. 2014; 11:1208-1217.
28 Hausner S H, Abbey C K, Bold R J, et al. Targeted in vivo imaging of integrin αvβ6 with an improved radiotracer and its relevance in a pancreatic tumor model. Cancer Res. 2009; 69:5843-5850.
29 Singh A N, McGuire M J, Li S, et al. Dimerization of a phage-display selected peptide for imaging of αvβ6-integrin: two approaches to the multivalent effect. Theranostics, 2014; 4:745-760.
30 Hausner S H, Bauer N, Sutcliffe J L. In vitro and in vivo evaluation of the effects of aluminum [¹⁸F]fluoride radiolabeling on an integrin αvβ6-specific peptide. Nucl Med Biol. 2014; 41:43-50.
31 Hausner S H, Bauer N, Hu L Y, Knight L M, Sutcliffe J L. The effect of bi-terminal PEGylation of an integrin αvβ6-targeted ¹⁸F-peptide on pharmacokinetics and tumor uptake. J Nucl Med. 2015; 56:784-790.
32 Hausner S H, Carpenter R D, Bauer N, Sutcliffe J L. Evaluation of an integrin αvβ6-specific peptide labeled with [¹⁸F]fluorine by copper-free, strain-promoted click chemistry. Nucl Med Biol. 2013; 233:233-239.
33 Flechsig P, Lindner T, Loktev A, Roesch S, Mier W, Sauter M, Meister M, Herold-Mende C, Haberkorn U, Altmann A. PET/CT Imaging of NSCLC with a αvβ6 Integrin-Targeting Peptide. Mol Imaging Biol 2019, DOI:10.1007/s11307-018-1296-6.
34 Hausner S H, Bold R J, Cheuy L Y, Chew H K, Daly M E, Davis R A, Foster C C, Kim E J, Sutcliffe J L. Preclinical Development and First-in-Human Imaging of the Integrin αvβ6 with [18F]αvβ6-Binding Peptide in Metastatic Carcinoma. Clin Cancer Res. 2019; DOI: 10.1158/1078-0432.
35 Kimura R H, et al. Evaluation of integrin αvβ6 cystine knot PET tracers to detect cancer and idiopathic pulmonary fibrosis. Nature Communications 2019, 10:4673 https://doi.org/10.1038/s41467-019-11863-w
36 Maltsev O V, Marelli U K, Kapp T G, et al. Stable peptides instead of stapled peptides: highly potent αvβ6-selective integrin ligands. Angew Chem Int Ed. 2016; 55:1535-1539.
37 Kapp T G, Kessler H, Maltsev O V. WO002017046416A1.
38 Notni J, Reich D, Maltsev O V, Kapp T G, Steiger K, Hoffmann F, Esposito I, Weichert W, Kessler H, Wester H J. In-vivo PET Imaging of the Cancer Integrin αvβ6 Using ⁶⁸Ga-Labeled Cyclic RGD Nonapeptides. J Nucl Med. 2017; 58:671-677.
39 Färber SF, Wurzer A, Reichart F, Beck R, Kessler H, Wester R I, Notni J. Therapeutic Radiopharmaceuticals Targeting Integrin αvβ6. ACS Omega 2018; 3:2428-2436.
40 Lattuada L, Barge A, Cravotto G, Giovenzana G B, Tei L. The synthesis and application of polyamino polycarboxylic bifunctional chelating agents. Chem Soc Rev 2011; 40:3019-3049.
41 Sipos B, Hahn D, Carceller A, et al Immunohistochemical screening for b6-integrin subunit expression in adenocarcinomas using a novel monoclonal antibody reveals strong up-regulation in pancreatic ductal adenocarcinomas in vivo and in vitro. Histopathology 2004, 45, 226-236.
42 Steiger K, Schlitter A M, Weichert W, Esposito I, Wester R I, Notni J. Perspective of αvβ6-Integrin Imaging for Clinical Management of Pancreatic Carcinoma and Its Precursor Lesions. Molecular Imaging 2017; 16:1-3.
43 Baranyai Z, Reich D, Vagner A, Weineisen M, TOth I, Wester R I, Notni J. A shortcut to high-affinity Ga-68 and Cu-64 radiopharmaceuticals: one-pot click chemistry trimerisation on the TRAP platform. Dalton Trans. 2015; 44:11137-11146.
44 Reichart F, Maltsev O V, Kapp T G, Räder, A F B, Weinmüller M, Marelli U K, Notni J, Wurzer A, Beck R, Wester R I, Steiger K, Di Maro S, Di Leva F S, Marinelli L, Nieberler M, Reuning U, Schwaiger M, Kessler H. Selective Targeting of Integrin αvβ8 by a Highly Active Cyclic Peptide. J Med Chem. 2019; 62:2024-2037.
45 Notni J, Simecek J, Hermann P, Wester H J. TRAP, a powerful and versatile framework for gallium-68 radiopharmaceuticals. Chem Eur J. 2011; 17:14718-14722.

1. A conjugate represented by the following formula (I)
E(Cp)ₙ (I)
wherein each Cp represents a cyclopeptide of the formula cyclo(YRGDLAYp(NMe)K), “Tyr₂”, n is an integer selected from 1 to 4, and E represents an effector moiety, wherein the effector moiety is covalently bonded to the cyclopeptide via the terminal amino group of the (NMe)K residue and wherein the effector moiety contains an atom or atomic group suitable for diagnosing, imaging or treating medical indications associated with increased expression of αvβ6-integrin, or a pharmaceutically acceptable salt, hydrate, solvate, ester or polymorph thereof.

2. The conjugate or pharmaceutically acceptable salt, hydrate, solvate, ester or polymorph thereof of claim 1, wherein the conjugate is selected from the following group of structures:
E(Tyr₂)₁,E(Tyr₂)₂,E(Tyr₂)₃,E(Tyr₂)₄.

3. The conjugate or pharmaceutically acceptable salt, hydrate, solvate, ester or polymorph thereof of claim 1, wherein the conjugate of formula (I) is characterized by a formula selected from the following formulae (Ia), (Ia′), (Ib) to (If):
Aa(Cg)(SCp)ₙ (Ia)
Aa′(Cg)ₖ(SCp)ₙ (Ia′)
Aa(Cg)ₖ(SCp)_{n′}(SAa′) (Ib)
Aa′(Cm)(SCp)ₙ (Ic)
(Cm)(SCp)ₙ₋ₒ(S(Aa′)ₚ(Cp)m)ₒ (Id)
(Cm)(SCp)ₙ₋ₒ(SCp(Aa′)ₚ)ₒ (Ie)
Cp(Aa′)ₚ (If)
wherein Aa stands for an active atom or active atomic group capable of forming a chelate complex, Aa′ stands for an active atom or active atomic group capable of forming a covalent bond, Cg stands for a chelating group, k is 1 or 0, S stands for an atomic group acting as a spacer and n is as defined above with respect to formula (I) with the proviso that n is 1 if k is 0, o can be any integer from 1 to n, p can be 1 or 2, m is 0 or 1, n′ is 1, 2 or 3 with the proviso that n′+1 is the number of free valences of the chelating group or less and Cm is a central moiety comprising 1 to 30 atoms selected from C, N, O, S and P.

4. The conjugate or pharmaceutically acceptable salt, hydrate, solvate, ester or polymorph thereof of claim 3, wherein the active atom or active atomic group is selected from a radioisotope suitable for scintigraphy, SPECT or PET imaging, or targeted radiotherapy; a chromophore of a fluorescence dye, a contrast agent for magnetic resonance imaging, an atom or atomic group suitable for imaging by X-ray based technology, or an atom or atomic group derived from a therapeutic agent suitable for treating medical indications associated with increased expression of αvβ6-integrin, wherein the term “derived from” indicates that an atomic group contained in the conjugate has the same structure as the compound from which it is derived, the only difference being the replacement of a hydrogen atom by a covalent bond for binding the atomic group to the remainder of the conjugate.

5. The conjugate or pharmaceutically acceptable salt, hydrate, solvate, ester or polymorph thereof of claim 3, wherein the active atom or active atomic group is a metal ion selected from La³⁺, Ce³⁺, Pr³⁺, Nd³⁺, Sm³⁺, Eu²⁺, Gd³⁺, Tb³⁺, Dy³⁺, Ho³⁺, Er³⁺, Tm³⁺, Yb³⁺, Lu³⁺, Sc³⁺, Y³⁺, Ga³⁺, Fe³⁺, Co²⁺, Co³⁺, Ge⁴⁺, In³⁺, Sn²⁺, Sn⁴⁺, Bi³⁺, Rh³⁺, Ru³⁺, Ru⁴⁺, Ag⁺, Au³⁺, Pb²⁺, Pd²⁺, Pd⁴⁺, Pm³⁺, Ac³⁺, Ti⁴⁺, Zr⁴⁺ Al³⁺, Cr³⁺, Cu²⁺, Zn²⁺ and mixtures thereof.

6. The conjugate or pharmaceutically acceptable salt, hydrate, solvate, ester or polymorph thereof of claim 3, wherein the active atom or active atomic group is a radioisotope selected from ⁴³Sc, ⁴⁴Sc, ⁴⁶Sc, ⁴⁷Sc, ⁵⁵Co, ^{99m}Tc, ²⁰³Pb, ²¹²Pb, ⁶⁶Ga, ⁶⁷Ga, ⁶⁸Ga, ⁷²As, ¹¹¹In,^{113m}In, ^{114m}In, ⁹⁷Ru, ⁶²Zn, ⁶¹Cu, ⁶²Cu, ⁶⁴Cu, ⁵²Fe, ^{52m}Mn, ⁵¹Cr, ¹⁸⁶Re, ¹⁸⁸Re, ⁷⁷As, ⁸⁶Y, ⁹⁰Y, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁵²Tb, ¹⁵⁵Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ⁸⁸Zr, ⁸⁹Zr, ²¹²Bi, ²¹³Bi, ²²⁵Ac, and mixtures hereof.

7. The conjugate or pharmaceutically acceptable salt, hydrate, solvate, ester or polymorph thereof of claim 3, wherein the active atom or active atomic group is a non-metal radioisotope selected from ¹¹C, ¹³N, ¹⁵O, ¹⁸F, ¹²³I, ¹²⁴I, ¹²⁵I, ¹³¹I.

8. The conjugate or pharmaceutically acceptable salt, hydrate, solvate, ester or polymorph thereof of claim 3, wherein the active atom or active atomic group is a contrast agent for magnetic resonance imaging selected from Gd, Fe, and Mn.

9. The conjugate or pharmaceutically acceptable salt, hydrate, solvate, ester or polymorph thereof of claim 3, wherein the active atom or active atomic group is a therapeutic group derived from a drug for the treatment of fibrosis or an anticancer drug selected from alkylating agents, anti-metabolites, anthracyclines, plant alkaloids, topoisomerase inhibitors and other anti-tumor drugs, wherein the term “derived from” indicates that an atomic group contained in the conjugate has the same structure as the compound from which it is derived, the only difference being the replacement of a hydrogen atom by a covalent bond for binding the atomic group to the remainder of the conjugate.

10. The conjugate or pharmaceutically acceptable salt, hydrate, solvate, ester or polymorph thereof of claim 3, wherein the atomic group acting as a spacer is a linear chain of 2 to 20 and preferably 3 to 10 atoms selected from C, N, O, P and S, which optionally carry one or more substituents, the remaining valences being saturated by hydrogen.

11. The conjugate or pharmaceutically acceptable salt, hydrate, solvate, ester or polymorph thereof of claim 3, wherein the atomic group acting as a spacer is selected from the following formulae (IIIa) to (IIIf):
*—C(O)—(CH₂)ₖ-(taz)ₗ-(CH₂)ₘ₋ (IIIa)
*—C(O)—(CH₂)ₖ—NH—CO—(CH₂)ₘ₋ (IIIb)
*—C(O)—(CH₂)ₖ—CO—NH—(CH₂)ₘ₋ (IIIc)
*—C(O)—(CH₂)ₖ-(taz)ₗ-(CH₂)ₒ—CO—NH—(CH₂)ₘ₋ (IIId)
*—C(O)—(CH₂)ₖ-(taz)ₗ-(CH₂)ₒ—NH—CO—(CH₂)ₘ₋ (IIIe)
*—C(O)—(CH₂)ₖ—CO—NH—(CH₂)ₒ-(taz)ₗ-(CH₂)ₘ₋ (IIIf)
*—C(O)—(CH₂)ₖ—NH—CO—(CH₂)ₒ-(taz)ₗ-(CH₂)ₘ₋ (IIIf)
wherein taz stands for a triazole ring with all three nitrogen atoms being adjacent to each other, 1 may be 0 or 1, k, m and, if present, o are integers independently selected from the range of 0 to 20 such that k+m=2-20 and k+m+o=2-20, respectively, and wherein the asterisk (*) marks the point of attachment of the cyclopeptide.

12. The conjugate or pharmaceutically acceptable salt, hydrate, solvate, ester or polymorph thereof of claim 3, wherein the chelating group is selected from the following formulae (IVa) to (IVd): 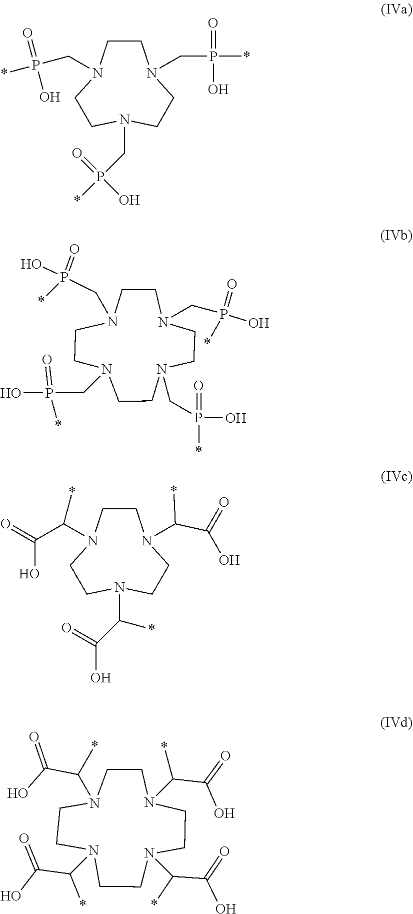
wherein the asterisk (*) marks the point of attachment of the atomic group acting as a spacer, with the proviso that if the number of cyclopeptides and associated spacers (as characterized by variable n) is less than the number of valences of the chelating group, the remaining valences shown by the asterisk are saturated by hydrogen or another atomic group, preferably a group selected from —CH₂—COOH and —CH₂—CH₂—COOH.

13. The conjugate or pharmaceutically acceptable salt, hydrate, solvate, ester or polymorph thereof of claim 1, wherein the conjugate contains a structure selected from compounds C-1 to C-4, C-7, C-10 to C-12, C-15 to C-18, C-23, and C-24 as specified in the description.

14. The conjugate or pharmaceutically acceptable salt, hydrate, solvate, ester or polymorph thereof of claim 1 for use in a method for diagnosing or imaging a disease associated with increased expression of αvβ6-integrin, preferably fibrosis or cancer.

15. The conjugate or pharmaceutically acceptable salt, hydrate, solvate, ester or polymorph thereof of claim 1 for use in a method of treating a disease associated with increased expression of αvβ6-integrin, preferably fibrosis or cancer.

16. A method of localizing cells with increased expression of αvβ6-integrin within a patient, wherein a conjugate or pharmaceutically acceptable salt, hydrate, solvate, ester or polymorph thereof of claim 1 has been administered to the patient, wherein the method comprises subjecting the patient to an imaging method selected from PET, SPECT, MRI, and X-ray computed tomography, wherein the conjugate contains an active atom or atomic group that is matched with the imaging method to be carried out.

17. A building block compound selected from compounds of formula (IIa):
Cg(SCp)ₙ (IIa)
wherein Cg stands for a chelating group, S stands for an atomic group acting as a spacer, each Cp is a cyclopeptide of the formula cyclo(YRGDLAYp(NMe)K), and n is an integer of from 1 to 4;
cyclo(YRGDLAYp(NMe)K); cyclo(3-I-YRGDLAYp(NMe)K); cyclo(3-I-YRGDLA3-I-Yp(NMe)K); cyclo(YRGDLA3-I-Yp(NMe)K);
wherein 3-I-Y represents a Tyr residue that carries an iodine atom in the 3-position of the phenyl ring, wherein said iodine atom can be any non-radioactive isotope or radioisotope of iodine;

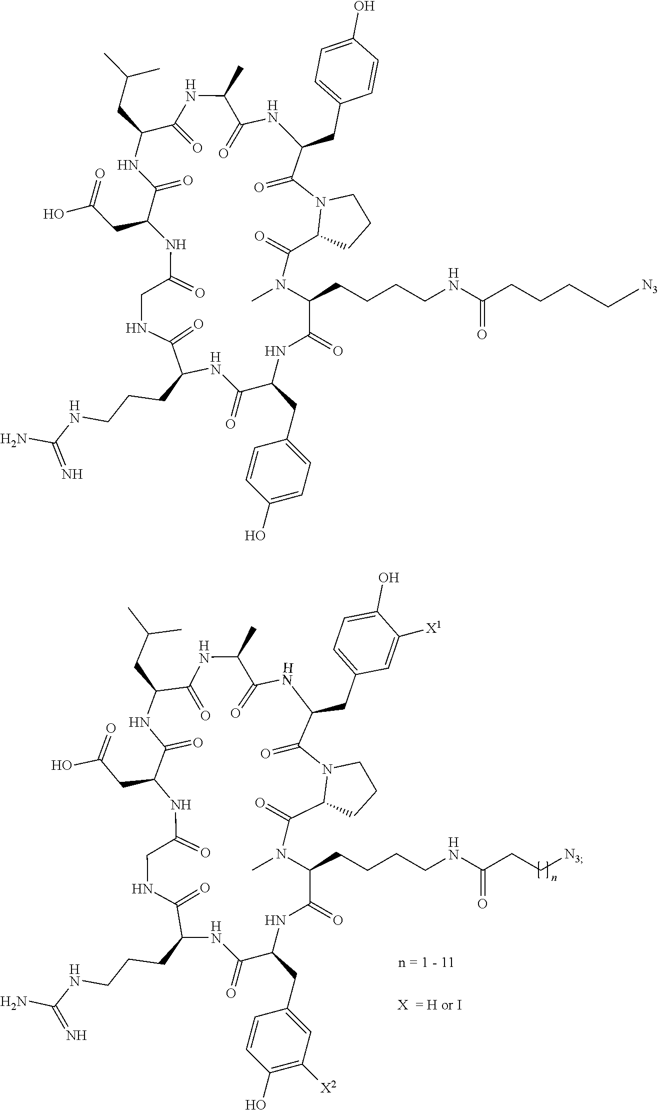

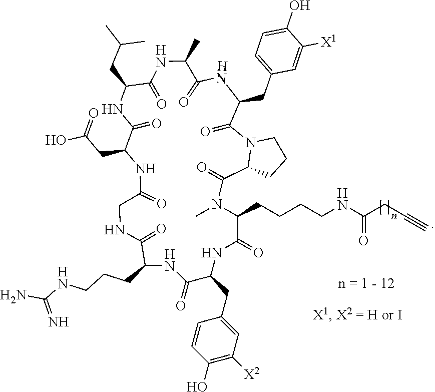

18. A pharmaceutical composition comprising the conjugate or a pharmaceutically acceptable salt, hydrate, solvate, ester or polymorph thereof of claim 1 and one or more pharmaceutically acceptable excipients and optionally one or more other therapeutic agents.

19. A method of diagnosing or imaging a disease associated with increased expression of αvβ6-integrin comprising administering to a subject in need thereof a therapeutically effective amount of the conjugate or pharmaceutically acceptable salt, hydrate, solvate, ester or polymorph thereof of claim 1, wherein the disease associated with increased expression of αvβ6-integrin is preferably fibrosis or cancer.

20. A method of treating a disease associated with increased expression of αvβ6-integrin comprising administering to a subject in need thereof a therapeutically effective amount of the conjugate or pharmaceutically acceptable salt, hydrate, solvate, ester or polymorph thereof of claim 1, wherein the disease associated with increased expression of αvβ6-integrin is preferably fibrosis or cancer.
